(19) United States
(12) Patent Application Publication
(10) Pub. No.: US 2004/0091857 A1
(43) Pub. Date: May 13, 2004

(54) Gene expression profiling

(76) Inventors: Girish N. Nallur, Guilford, CT (US); Chenghua Luo, Waterford, CT (US); Kajal Chowdhury, Hamden, CT (US); Robert Pinard, New Haven, CT (US)

Correspondence Address: NEEDLE & ROSENBERG, P.C., SUITE 1000, 999 PEACHTREE STREET, ATLANTA, GA, 30309-3915, US

(21) Appl. No.: 09/910,383

(22) Filed: Jul. 20, 2001

(51) Int. Cl. C12Q 1/68; C12P 19/34

(52) U.S. Cl. 435/6; 435/91.2

(57) ABSTRACT

Disclosed are methods and compositions for manipulating and detecting nucleic acids. The method generally involves association of a rolling circle replication primer with a cDNA strand. Preferred forms of the methods involve replicating one or more amplification target circles to produce one or more tandem sequence DNAs. Such replication is referred to as rolling circle replication. Preferably, each tandem sequence DNA is coupled to a rolling circle replication primer and the rolling circle replication primer is associated with a cDNA strand. In some embodiments the rolling circle replication primer comprises a capture tag and the association occurs via the capture tag. In some embodiments the cDNA strand is hybridized to a capture probe. Preferably, the cDNA strand comprises an RT primer, wherein the cDNA strand is produced by reverse transcribing a nucleic acid sample with the RT primer.

### FIELD OF THE INVENTION

The present invention is in the field of nucleic acid manipulation and detection, and specifically in the area of manipulating and detecting particular nucleic acid molecules.

### BACKGROUND OF THE INVENTION

Numerous nucleic acid amplification techniques have been devised, including strand displacement cascade amplification (SDCA)(referred to herein as exponential rolling circle amplification (ERCA)) and rolling circle amplification (RCA)(U.S. Pat. No. 5,854,033; PCT Application No. WO 97/19193; Lizardi et al., Nature Genetics 19(3):225-232 (1998)); multiple displacement amplification (MDA)(PCT Application WO 99/18241); strand displacement amplification (SDA)(Walker et al., Nucleic Acids Research 20:1691-1696 (1992), Walker et al., Proc. Natl. Acad. Sci. USA 89:392-396 (1992)); polymerase chain reaction (PCR) and other exponential amplification techniques involving thermal cycling, self-sustained sequence replication (3SR), nucleic acid sequence based amplification (NASBA), and amplification with Qβ replicase (Birkenmeyer and Mushahwar, J. Virological Methods 35:117-126 (1991); Landegren, Trends Genetics 9:199-202 (1993)); and various linear amplification techniques involving thermal cycling such as cycle sequencing (Craxton et al., Methods Companion Methods in Enzymology 3:20-26 (1991)). Amplification of a sequence corresponding to an RNA molecule is generally accomplished by first generating a cDNA which is then amplified using standard procedures to generate DNA molecules. For instance, in the commonly used RT-PCR method of amplifying nucleic acid sequence derived from mRNA, a DNA molecule is produced from an RNA template using reverse transcriptase. The resultant DNA molecule is then amplified.

Rolling Circle Amplification (RCA) driven by DNA polymerase can replicate circular oligonucleotide probes with either linear or geometric kinetics under isothermal conditions (Lizardi et al., Nature Genet. 19:225-232 (1998); U.S. Pat. Nos. 5,854,033 and 6,143,495; PCT Application No. WO 97/19193). If a single primer is used, RCA generates in a few minutes a linear chain of hundreds or thousands of tandemly-linked DNA copies of a target that is covalently linked to that target. Generation of a linear amplification product permits both spatial resolution and accurate quantitation of a target. DNA generated by RCA can be labeled with fluorescent oligonucleotide tags that hybridize at multiple sites in the tandem DNA sequences. RCA can be used with fluorophore combinations designed for multiparametric color coding (PCT Application No. WO 97/19193), thereby markedly increasing the number of targets that can be analyzed simultaneously. RCA technologies can be used in solution, in situ and in microarrays. In solid phase formats, detection and quantitation can be achieved at the level of single molecules (Lizardi et al., 1998). Ligation-mediated Rolling Circle Amplification (LM-RCA) involves circularization of a probe molecule hybridized to a target sequence and subsequent rolling circle amplification of the circular probe (U.S. Pat. Nos. 5,854,033 and 6,143,495; PCT Application No. WO 97/19193).

Therefore, it is an object of the present invention to provide method and compositions for manipulating nucleic acid molecules so that they may more easily be observed and detected.

### BRIEF SUMMARY OF THE INVENTION

Disclosed are methods and compositions for manipulating and detecting nucleic acids. The method generally involves association of a rolling circle replication primer with a cDNA strand. Preferred forms of the methods involve replicating one or more amplification target circles to produce one or more tandem sequence DNAs. Such replication is referred to as rolling circle replication. Preferably, each tandem sequence DNA is coupled to a rolling circle replication primer and the rolling circle replication primer is associated with a cDNA strand. In some embodiments the rolling circle replication primer comprises a capture tag and the association occurs via the capture tag. In some embodiments the cDNA strand is hybridized to a capture probe. Preferably, the cDNA strand comprises an RT primer, wherein the cDNA strand is produced by reverse transcribing a nucleic acid sample with the RT primer.

Additional aspects and advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate several exemplary embodiments of the invention and together with the description, serve to explain the principles of the invention.

FIG. 1 shows one particular embodiment of the disclosed methods. The RT primer has a 5′ terminal biotin attached, the generated cDNA molecules hybridize to the capture probes on an array, immunoRCA (a form of indirect RCA) through an anti-biotin Ab conjugate is performed, and then utilize single color determination for detection.

FIG. 2 shows one embodiment of the disclosed methods wherein there is cDNA fragmentation and haptenylation with bio-ddNTP.

FIG. 3 shows one embodiment of the disclosed methods wherein reverse transcription is performed with allyl amine dUTP and the rolling circle amplification primer is coupled to the cDNA with an NHS ester.

FIG. 4 shows one embodiment of the disclosed methods wherein biotin ddNTP is incorporated into the cDNA during reverse transcription.

FIG. 5 shows one embodiment of the disclosed methods wherein biotin dNTP is incorporated into cDNA during reverse transcription. After hybridization to capture probes on the array, the biotin is detected by immunoRCA using anti-biotin antibody or neutravidin conjugated to an RCA primer. RCA is performed in the presence of a modified nucleotide triphosphate, namely BrdUTP, so that it is incorporated into the resulting RCA product (tandem sequence DNA). The RCA product is then detected with anti-BrdU-antibody conjugated to a fluorophore, such as phycoerythrin (PE).

### DETAILED DESCRIPTION OF THE INVENTION

The present invention may be understood more readily by reference to the following detailed description of preferred embodiments of the invention and the Examples included therein and to the Figures and their previous and following description.

Before the present compounds, compositions, articles, devices, and/or methods are disclosed and described, it is to be understood that this invention is not limited to specific synthetic methods or to specific reagents, as such may, of course, vary. It is also to be understood that the terminology used herein is for the purpose of describing particular embodiments only and is not intended to be limiting.

Reference will now be made in detail to the present preferred embodiment(s) of the invention, examples of which are illustrated in the accompanying drawings. Wherever possible, the same reference numbers are used throughout the drawings to refer to the same or like parts.

Disclosed are methods and compositions for manipulating and detecting nucleic acids. The method generally involves association of a rolling circle replication primer with a cDNA strand. Preferred forms of the methods involve replicating one or more amplification target circles to produce one or more tandem sequence DNAs. Such replication is referred to as rolling circle replication. Preferably, each tandem sequence DNA is coupled to a rolling circle replication primer and the rolling circle replication primer is associated with a cDNA strand. In some embodiments the rolling circle replication primer comprises a capture tag and the association occurs via the capture tag. In some embodiments the cDNA strand is hybridized to a capture probe. Preferably, the cDNA strand comprises an RT primer, wherein the cDNA strand is produced by reverse transcribing a nucleic acid sample with the RT primer.

These forms of the disclosed methods can also involve fragmented cDNA, wherein the fragmented cDNA strand is a fragment of a cDNA strand. In this case, the rolling circle replication primer is associated with the fragmented cDNA strand, and it is the fragmented cDNA that comprises the capture tag.

In some embodiments of these forms of the methods, it is the RT primer that comprises the capture tag through which the association occurs (rather than the rolling circle replication primer). In other embodiments the cDNA strand can comprise the capture tag through which the association occurs. In still other embodiments both the rolling circle replication primer and the RT primer comprise capture tags, or both the rolling circle replication primer and cDNA strand comprise capture tags, with the association occurring via one or both of the capture tags. In other embodiments, the RT primer can comprise a rolling circle replication primer portion and a reverse transcription primer portion, wherein the reverse transcription primer portion and the rolling circle replication primer portion each comprise a 5′ end, wherein the reverse transcription primer portion and the rolling circle replication primer portion are not linked via their 5′ ends.

The disclosed methods make use of rolling circle replication in various forms collectively referred to as Rolling Circle Amplification (RCA). RCA driven by DNA polymerase can replicate circular oligonucleotide probes with either linear or geometric kinetics under isothermal conditions (Lizardi et al., Nature Genet. 19: 225-232 (1998); U.S. Pat. Nos. 5,854,033 and 6,143,495; PCT Application No. WO 97/19193). If a single primer is used, RCA generates in a few minutes a linear chain of hundreds or thousands of tandemly-linked DNA copies of the circular molecule that are covalently linked to the primer. DNA generated by RCA can be labeled with fluorescent oligonucleotide tags that hybridize at multiple sites in the tandem DNA sequences. RCA can be used with fluorophore combinations designed for multiparametric color coding (PCT Application No. WO 97/19193), thereby markedly increasing the number of amplified molecules that can be analyzed simultaneously. RCA technologies can be used in solution, in situ and in microarrays. In solid phase formats, detection and quantitation can be achieved at the level of single molecules (Lizardi et al., 1998). Ligation-mediated Rolling Circle Amplification (LM-RCA) involves circularization of a probe molecule hybridized to a target sequence and subsequent rolling circle amplification of the circular probe (U.S. Pat. Nos. 5,854,033 and 6,143,495; PCT Application No. WO 97/19193).

### Methods

Disclosed are methods for manipulating and detecting nucleic acids. A preferred form of the methods is a method of using messenger RNA, the method comprising (a) mixing one or more RT primers with a nucleic acid sample and reverse transcribing to produce cDNA strands each comprising one of the RT primers, wherein each RT primer comprises a reverse transcription primer portion, (b) mixing the cDNA strands with a set of capture probes under conditions that promote hybridization of the cDNA strands to the capture probes, (c) mixing one or more rolling circle replication primers with the cDNA strands under conditions that promote association of the cDNA strands with the rolling circle replication primers, wherein the rolling circle replication primers each comprise a capture tag, and wherein the association occurs via the capture tag, (d) mixing one or more amplification target circles with the rolling circle replication primers under conditions that promote association of the rolling circle replication primers with the amplification target circles, (e) incubating the amplification target circles under conditions that promote replication of the amplification target circles, wherein replication of the amplification target circles results in the formation of tandem sequence DNA. Thus, generally the method includes manipulation of the base nucleic acid, interaction with some type of capture molecule (such as a capture probe), and amplification.

A. Manipulation of Base Nucleic Acid

One aspect of the disclosed methods involves the manipulation of a base nucleic acid to produce a manipulated product nucleic acid. This manipulation can occur through any known mechanism, such as reverse transcription or various DNA polymerase based techniques, such as PCR amplification. The base nucleic acid is typically a nucleic acid of interest or a nucleic acid that is somehow related to a nucleic acid of interest, such as a cDNA of an mRNA. “Base nucleic acid” is intended to refer to a nucleic acid prior to manipulation in the disclosed method. A “manipulated product nucleic acid” refers to the nucleic acid resulting from manipulation of a base nucleic acid in the disclosed method. For convenience, both base nucleic acids and manipulated product nucleic acids at times are referred to herein as “nucleic acids.” A preferred base nucleic acid is messenger RNA. A preferred manipulated product nucleic acid is a cDNA strand. In one preferred embodiment the step of manipulation of the base nucleic acid occurs through reverse transcription of a specific mRNA or an mRNA population. This step occurs by mixing one or more RT primers with a nucleic acid sample and reverse transcribing to produce cDNA strands each comprising one of the RT primers, wherein each RT primer comprises a reverse transcription primer portion.

1. Mixing

In a variety of steps of the disclosed method various reagents are mixed together. Typically this mixing indicates a physical mixing. Also typically, the mixing will occur such that various interactions or associations will occur. For example, when performing the step of mixing the RT primer with a target population of mRNA or a specific mRNA the mixing would typically occur such that the RT primer will hybridize to mRNA in the target mRNA population or to the specific mRNA so that extension of the primer can take place. Mixing may involve, but it is not limited to, stirring or other mechanical shuffling of the reagents. Thus, mixing includes merely bringing the reagents into contact.

2. Reverse Transcription

If the disclosed method includes performing enzymatic processing involving reverse transcription, it can be performed using any known method for performing a reverse transcription reaction.

In some embodiments of the disclosed methods a plurality of messenger RNA molecules are manipulated. The plurality of different messenger RNA molecules of interest may comprise a set of messenger RNA molecules derived from, or present in, a source of interest. Such a source can include, for example, cells, tissue or any other source of mRNA. The disclosed methods may also involve a plurality of different messenger RNA molecules which are associated with a condition or disease state of the cells, tissue, or the source of the mRNA sample. In some embodiments of the disclosed methods, the plurality of different messenger RNA molecules of interest comprises a set of messenger RNA molecules representing a catalog of messenger RNA molecules from a source of interest.

The disclosed methods also may include a plurality of different messenger RNA molecules of interest comprising a set of messenger RNA molecules from one or more of sources of interest. The messenger RNA molecules used in the method can be present in an mRNA sample. Messenger RNA samples are one form of nucleic acid sample. Thus, in some embodiments of the method a nucleic acid sample is reverse transcribed to produce cDNA strands.

B. Capture Probe Interaction

The disclosed methods also typically include some type interaction or capture the nucleic acids, such as interaction with a capture probe. Such an interaction, referred to as a capture probe interaction, can be used, for example, to sort, separate, or immobilize a base nucleic acid or the manipulated product nucleic acid. Capture probes are preferably immobilized in arrays. In such cases, interaction of the nucleic acids with capture probes is a form of array interaction. A capture probe interaction can involve various types of immobilizations or collections of the base nucleic acid or manipulated product nucleic acid. For example, in a preferred embodiment, the cDNA strands produced by reverse transcription can be mixed with a set of capture probes under conditions that promote hybridization of the cDNA strands to the capture probes. Capture probe interaction can be performed at any time, but preferably is performed prior to mixing the rolling circle primer with the molecules to be associated with the rolling circle primer.

It is understood that many different forms of substrate can be used including, for example, DNA chips or membranes. The disclosed methods preferably can be performed using capture probes that are immobilized on a substrate, preferably in an array. In some embodiments of the disclosed methods, the capture probes are immobilized via a capture tag coupled to the capture probes.

Preferably, each capture probe comprises a sequence matching all or a portion of the sequence of messenger RNA molecules of interest. In some embodiments of the disclosed methods a set of capture probes collectively comprises sequence matching all or a portion of the sequence of a plurality of different messenger RNA molecules of interest.

Hybridization of nucleic acids is well understood in the art. A given sequence will hybridize to its complement with a particular affinity which is controlled by many factors including temperature, salt concentrations, and pH.

By “probe,” “primer,” or oligonucleotide is meant a single-stranded DNA or RNA molecule of defined sequence that can base-pair to a second DNA or RNA molecule that contains a complementary sequence. The stability of the resulting hybrid depends upon the extent of the base-pairing that occurs. The extent of base-pairing is affected by parameters such as the degree of complementarity between the probe and complementary sequences and the degree of stringency of the hybridization conditions. The degree of hybridization stringency is affected by parameters such as temperature, salt concentration, and the concentration of organic molecules such as formamide, and is determined by methods known to one skilled in the art. Probes or primers specific for a given nucleic acid (for example, genes and/or mRNAs) have at least 80%-90% sequence complementarity, preferably at least 91%-95% sequence complementarity, more preferably at least 96%-99% sequence complementarity, and most preferably 100% sequence complementarity to the region of the nucleic acid to which they hybridize. Probes, primers, and oligonucleotides may be detectably-labeled, either radioactively, or non-radioactively, by methods well-known to those skilled in the art. Probes, primers, and oligonucleotides are used for methods involving nucleic acid hybridization, such as: nucleic acid sequencing, reverse transcription and/or nucleic acid amplification by the polymerase chain reaction, single stranded conformational polymorphism (SSCP) analysis, restriction fragment polymorphism (RFLP) analysis, Southern hybridization, Northern hybridization, in situ hybridization, electrophoretic mobility shift assay (EMSA).

By “specifically hybridizes” is meant that a probe, primer, or oligonucleotide recognizes and physically interacts (that is, base-pairs) with a substantially complementary nucleic acid under high stringency conditions, and does not substantially base pair with other nucleic acids.

By “high stringency conditions” is meant conditions that allow hybridization comparable with that resulting from the use of a DNA probe of at least 40 nucleotides in length, in a buffer containing 0.5 M NaHPO₄, pH 7.2, 7% SDS, 1 mM EDTA, and 1% BSA (Fraction V), at a temperature of 65° C., or a buffer containing 48% formamide, 4.8×SSC, 0.2 M Tris-Cl, pH 7.6, 1×Denhardt's solution, 10% dextran sulfate, and 0.1% SDS, at a temperature of 42° C. Other conditions for high stringency hybridization, such as for PCR, Northern, Southern, or in situ hybridization, DNA sequencing, etc., are well-known by those skilled in the art of molecular biology. See, for example, F. Ausubel et al., Current Protocols in Molecular Biology, John Wiley & Sons, New York, N.Y., 1998.

The disclosed methods also contemplate capture probes which are extendable when a cDNA strand is hybridized to the capture probe and where the ends of the capture probes are designed to be extendable only when a cDNA strand corresponding to a particular form of a messenger RNA of interest is hybridized to the capture probe. In other embodiments, the capture probes are not extendable by polymerase.

In some embodiments of the methods an additional step can be performed. This step involves mixing one or more sub-probes with the cDNA strands, wherein each sub-probe is designed to hybridize to a cDNA strand adjacent to where a capture probe hybridizes, ligating sub-probes and capture probes hybridized to cDNA strands. This step can additionally be performed following ligation by incubating the capture probes at a temperature above the melting temperature of the capture probe but below the melting temperature of the ligated capture probe/sub-probe. This variation of the disclosed methods allows for an increase in the specificity with which the capture probe(s) associate with the molecules they are supposed to associate with. This step, as with capture probe interaction, can be performed at any time, but preferably is performed prior to mixing the rolling circle primer with the molecules to be associated with the rolling circle primer.

When capture probes and sub-probes hybridize to cDNA strands, the probes can either be hybridized such that the ends of the probes are immediately juxtaposed or such that there is a gap between the two ends. In order to join the two probes, this gap space must be bridged. The gap space formed by a capture probe and sub-probe hybridized to a nucleic acid is normally occupied by one or more gap oligonucleotides as described herein. Such a gap space may also be filled in by a gap-filling DNA polymerase prior to or during ligation. As an alternative, the gap space can be partially bridged by one or more gap oligonucleotides, with the remainder of the gap filled using DNA polymerase. This modified ligation operation is referred to herein as gap-filling ligation. The principles and procedure for gap-filling ligation are generally analogous to the filling and ligation performed in gap LCR (Wiedmann et al., PCR Methods and Applications (Cold Spring Harbor Laboratory Press, Cold Spring Harbor Laboratory, NY, 1994) pages S51-S64; Abravaya et al., Nucleic Acids Res., 23(4):675-682 (1995); European Patent Application EP0439182 (1991)). Gap-filling ligation provides a means for discriminating between closely related target sequences. Gap-filling ligation can be accomplished by using a DNA polymerase, referred to herein as a gap-filling DNA polymerase, that is different from the polymerase sued for amplification. Suitable gap-filling DNA polymerases are described elsewhere herein. Alternatively, DNA polymerases in general can be used to fill the gap when a stop base is used. The use of stop bases in the gap-filling operation of LCR is described in European Patent Application EP0439182. The principles of the design of gaps and the ends of flanking probes to be joined, as described in EP0439182, is generally applicable to the design of the gap spaces and the ends of target probe portions described herein. Gap-filling ligation is further described in U.S. Pat. No. 6,143,495.

C. Rolling Circle Platform Preparation

The disclosed methods also typically include a step or steps for amplifying the manipulated product nucleic acid. One form of that amplification is the use of rolling circle amplification as described elsewhere herein. When this method of amplification is used, a rolling circle platform must be prepared. The rolling circle platform comprises the primer needed for rolling circle replication, referred to herein as a rolling circle replication primer. In the disclosed methods, the rolling circle replication platform is associated with a target molecules, typically manipulated product nucleic acids or base nucleic acids. When the method involves reverse transcription and preparation of cDNA then typically this platform can be prepared by mixing one or more rolling circle replication primers with the cDNA strands under conditions that promote association of the cDNA strands with the rolling circle replication primers. Preferably, the rolling circle replication primers each comprise a capture tag, and wherein the association occurs via the capture tag.

It is understood, however that the rolling circle primer could be associated with whatever product arises from the manipulation of the base nucleic acid. For example, the rolling circle primer could associate with a PCR product of a DNA polymerase manipulation or with the mRNA of a transcription reaction.

1. Association

The association of the rolling circle primer with a target molecule (typically a base nucleic acid or manipulated product nucleic acid of the method) can occur through any of a variety of covalent and non-covalent mechanisms. For example, the association could occur through nucleic acid to nucleic acid interactions, protein to nucleic acid interactions, and protein to protein interactions. The association could also occur through covalent bond formation, such as the formation of a disulfide bond.

When the association is non-covalent, one way of qualifying the association is through the affinity between the rolling circle primer and the target molecule. For example, in some embodiments the association between the rolling circle replication primer and the target molecule can have a dissociation constant of less than or equal to 10⁻⁵ μM, or 10⁻⁶ μM, 10⁻⁷ μM, 10⁻⁸ μM, 10⁻⁹ μM, 10⁻¹⁰ μM, 10⁻¹¹ μM, 10⁻¹² μM, 10⁻¹³ μM, 10⁻¹⁴ μM, 10⁻¹⁵ μM, or 10⁻¹⁶ μM.

It is preferred that the association between the rolling circle replication primer and the target molecule is such that the rolling circle primer remains associated with the target molecule long enough for rolling circle amplification to take place and/or long enough for detection of the association.

In certain embodiments of the disclosed methods the association of the various components involved in the method can occur through capture tags. Capture tags are discussed in detail elsewhere herein, but for example the RT primer can comprise a capture tag or the cDNA can contain a capture tag or the manipulated product nucleic acid can contain a capture tag. The capture tag can comprise, for example, biotin, digoxigenin, bromodeoxyuridine, or any other hapten.

In a preferred forms of the disclosed method, the association occurs via a capture tag that is part of, or attached to, a rolling circle replication primer. The capture tag can associate with, for example, an RT primer or cDNA strand. A capture tag can also be a part of, or attached to, an RT primer. In this case, the association can occur via the capture tag on the RT primer, a capture tag on the rolling circle replication primer, or both. A capture tag can also be a part of, or attached to, a cDNA strand (or other manipulated product nucleic acid). In this case, the association can occur via the capture tag on the cDNA, a capture tag on the rolling circle replication primer, or both. A capture tag need not be a part of, or attached to, a rolling circle replication primer when a capture tag is part of, or attached to, the RT primer or cDNA strand.

In certain embodiments there can be more than one capture tag part of, or attached to, one or more of the molecules involved in the method and these capture tags may or may not specifically interact with each other. For example, the capture tag can be an antibody that interacts with biotin.

D. Rolling Circle Amplification

The disclosed methods generally include a step or steps of amplification that typically involves rolling circle amplification. When rolling circle amplification is involved the rolling circle replication primer and the rolling circle template must be associated together. This typically can occur through mixing one or more amplification target circles with the rolling circle replication primers under conditions that promote association of the rolling circle replication primers with the amplification target circles. To get replication of the amplification target circle the amplification target circle and the rolling circle replication primer typically are incubated under conditions that promote replication of the amplification target circles, wherein replication of the amplification target circles results in the formation of tandem sequence DNA. There are numerous variations of rolling circle amplification that can be used in the disclosed methods. In some embodiments the tandem sequence DNA can itself be replicated or otherwise amplified.

Following amplification, the amplified sequences can be detected and quantified using any of the conventional detection systems for nucleic acids such as detection of fluorescent labels, enzyme-linked detection systems, antibody-mediated label detection, and detection of radioactive labels.

Rolling circle amplification has two features that provide simple and consistent amplification and detection of a target nucleic acid sequence. First, target sequences are amplified via a small diagnostic probe with an arbitrary primer binding sequence. This allows consistency in the priming and replication reactions, even between probes having very different target sequences. Second, amplification takes place not in cycles, but in a continuous, isothermal replication: rolling circle replication. This makes amplification less complicated and much more consistent in output.

1. The Amplification Operation

The disclosed method includes a rolling circle amplification operation. Rolling circle amplification involves rolling circle replication of a circular DNA template molecule. A preferred circular DNA template molecule an amplification target circle (ATC). The amplification target circle can either be pre-formed prior to its use in the disclosed method, or it can be formed through ligation of an open circle probe as part of the method. Amplification target circles serve as substrates for a rolling circle replication. In addition to an amplification target circle, rolling circle replication uses a rolling circle replication primer and DNA polymerase. The DNA polymerase catalyzes primer extension and strand displacement in a processive rolling circle polymerization reaction that proceeds as long as desired, generating a molecule of 100,000 nucleotides or more that contains up to approximately 1000 tandem copies or more of a sequence complementary to the amplification target circle or open circle probe. This is referred to as tandem sequence DNA (TS-DNA).

During rolling circle replication one may additionally include radioactive, or modified nucleotides such as bromodeoxyuridine triphosphate, in order to label the DNA generated in the reaction. Alternatively, one may include suitable precursors that provide a binding moiety such as biotinylated nucleotides (Langer et al. (1981)). Unmodified TS-DNA can be detected using any nucleic acid detection technique.

The amplification operation can include additional nucleic acid replication or amplification processes. For example, TS-DNA can itself be replicated to form secondary TS-DNA. This process is referred to as secondary DNA strand displacement. The combination of rolling circle replication and secondary DNA strand displacement is referred to as linear rolling circle amplification (LRCA). The secondary TS-DNA can itself be replicated to form tertiary TS-DNA in a process referred to as tertiary DNA strand displacement. Secondary and tertiary DNA strand displacement can be performed sequentially or simultaneously. When performed simultaneously, the result is strand displacement cascade amplification. The combination of rolling circle replication and strand displacement cascade amplification is referred to as exponential rolling circle amplification (ERCA). Secondary TS-DNA, tertiary TS-DNA, or both can be amplified by transcription.

After RCA, a round of ligation-mediated RCA (LM-RCA) can be performed on the TS-DNA produced in the first RCA. LM-RCA is performed with an open circle probe, having target probe portions complementary to a target sequence in the TS-DNA produced in the first RCA. LM-RCA can also be performed on ligated OCPs or ATCs that have not been amplified. In this case, LM-RCA can be carried out on ATCs. Various forms of LM-RCA are described in U.S. Pat. No. 6,143,495.

E. Detection of Amplification Products

The association of the TS-DNA or other amplified DNA with the original nucleic acid to be manipulated (that is, the base nucleic acid) or the nucleic acid that may result from the manipulation (that is, the manipulated product nucleic acid) can be detected. The amplified nucleic acid typically can be detected following rolling circle replication. The amplified sequences can be detected using combinatorial multicolor coding probes (or other multiplex detection system) that allow separate and simultaneous detection of multiple different amplified ATCs associated with multiple different nucleic acid molecules. Major advantages of this method are that a large number of distinct nucleic acid molecules can be detected simultaneously, and that differences in the amounts of the various nucleic acid molecules in a sample can be accurately quantified.

Products of the amplification operation can be detected using any nucleic acid detection technique. Many techniques are known for detecting nucleic acids. Several preferred forms of detection are described elsewhere herein. The nucleotide sequence of the amplified sequences also can be determined using any suitable technique.

In certain embodiments the disclosed methods further comprise detecting the tandem sequence DNA, wherein detection of tandem sequence DNA indicates that the corresponding messenger RNA molecule was present in the nucleic acid sample. For example, in certain embodiments the tandem sequence DNA is detected while in association with the capture probes or the identity of the capture probe associated with a tandem sequence DNA indicates the identity of the corresponding messenger RNA molecule. In other embodiments, the tandem sequence DNA is detected at the site where the capture probe is located, and wherein the location of the capture probe indicates the identity of the corresponding messenger RNA molecule. Still further embodiments are where the detection is mediated by detection probes or by a detection label incorporated in the tandem sequence DNA. In certain embodiments, the detection label can be a ligand, for example where the ligand is biotin.

The disclosed methods also can further comprise mixing a set of detection probes with the tandem sequence DNA under conditions that promote hybridization between the tandem sequence DNA and the detection probes, and detecting a plurality of different sequences present in the tandem sequence DNA. In some embodiments, the tandem sequence DNA is collapsed using collapsing probes. In certain embodiments, the tandem sequence DNA is collapsed by mixing the collapsing probes with the tandem sequence DNA, and incubating under conditions that promote hybridization between the collapsing probes and the tandem sequence DNA.

In certain embodiments the disclosed method can further comprise, prior to or simultaneous with the mixing of the collapsing probes with the tandem sequence DNA, mixing detection probes with the tandem sequence DNA, and incubating under conditions that promote hybridization between the detection probes and the tandem sequence DNA. Also disclosed are methods where the collapsing probes comprise ligands, haptens, or both coupled to or incorporated into oligonucleotides.

A number of general concepts regarding detection and manipulation of nucleic acids are applicable to detection of the amplified products of the disclosed methods.

1. Primary Labeling

Primary labeling consists of incorporating labeled moieties, such as fluorescent nucleotides, biotinylated nucleotides, digoxygenin-containing nucleotides, or bromodeoxyuridine, during rolling circle amplification. For example, one may incorporate cyanine dye UTP analogs (Yu et al. (1994)) at a frequency of 4 analogs for every 100 nucleotides. A preferred method for detecting nucleic acid amplified in situ is to label the DNA during amplification with bromodeoxyuridine (BrdUrd or BrdU), followed by binding of the incorporated BrdU with a biotinylated anti-BrdU antibody (Zymed Labs, San Francisco, Calif.), followed by binding of the biotin moieties with Streptavidin-Peroxidase (Life Sciences, Inc.), and finally development of fluorescence with Fluorescein-tyramide (DuPont de Nemours & Co., Medical Products Dept.).

2. Secondary Labeling

Secondary labeling consists of using suitable molecular probes, such as detection probes, to detect the amplified nucleic acids. For example, an amplification target circle may be designed to contain several repeats of a known arbitrary sequence, referred to as detection tags. A secondary hybridization step can be used to bind detection probes to these detection tags. The detection probes may be labeled as described elsewhere herein with, for example, an enzyme, fluorescent moieties, or radioactive isotopes. By using three detection tags per ATC, and four fluorescent moieties per each detection circle, one may obtain a total of twelve fluorescent signals for every ATC repeat in the TS-DNA, yielding a total of 12,000 fluorescent moieties for every ligated open circle probe that is amplified by RCA.

3. Multiplexing and Hybridization Array Detection with ATCs

RCA is easily multiplexed by using sets of different amplification target circles, each set carrying different primer complement portions designed for binding to unique rolling circle replication primers and/or different spacer sequences designed for binding to unique address probes and/or unique detection probes. Note that although the primer complement portion of each ATC are different, the detection tag sequence and/or address tag sequence may remain unchanged, and thus the detection probe sequence and/or address probe sequence can remain the same for all TS-DNA. Only those amplification target circles that find their cognate rolling circle replication primer will give rise to TS-DNA.

The TS-DNA molecules generated by RCA are of high molecular weight and low complexity; the complexity being the length of the amplification target circle. There are two alternatives preferred for detecting a given TS-DNA. One is to include within the spacer region of the amplification target circle a unique detection tag sequence for each unique amplification target circle. TS-DNA generated from a given amplification target circle will then contain sequences corresponding to a specific detection tag sequence. A second alternative is to use the primer complement sequence present on the TS-DNA as the detection tag.

4. Combinatorial Multicolor Coding

A preferred form of multiplex detection involves the use of a combination of labels that either fluoresce at different wavelengths or are colored differently. One of the advantages of fluorescence for the detection of hybridization probes is that several labeled molecules can be visualized simultaneously in the same sample. Using a combinatorial strategy, many more molecules can be discriminated than the number of spectrally resolvable fluorophores. Combinatorial labeling provides the simplest way to label probes in a multiplex fashion since a probe fluor is either completely absent (−) or present in unit amounts (+); image analysis is thus more amenable to automation, and a number of experimental artifacts, such as differential photobleaching of the fluors and the effects of changing excitation source power spectrum, are avoided.

The combinations of labels establish a code for identifying different detection probes and, by extension, different nucleic acid molecules to which those detection probes are associated with. This labeling scheme is referred to as Combinatorial Multicolor Coding (CMC). Such coding is described by Speicher et al., Nature Genetics 12:368-375 (1996). Use of CMC in connection with rolling circle amplification is described in U.S. Pat. No. 6,143,495. Any number of labels, which when combined can be separately detected, can be used for combinatorial multicolor coding. It is preferred that 2, 3, 4, 5, or 6 labels be used in combination. It is most preferred that 6 labels be used. The number of labels used establishes the number of unique label combinations that can be formed according to the formula 2^{N}−1, where N is the number of labels. According to this formula, 2 labels forms three label combinations, 3 labels forms seven label combinations, 4 labels forms 15 label combinations, 5 labels form 31 label combinations, and 6 labels forms 63 label combinations.

For combinatorial multicolor coding, a group of different detection probes are used as a set. Each type of detection probe in the set is labeled with a specific and unique combination of fluorescent labels. For those detection probes assigned multiple labels, the labeling can be accomplished by labeling each detection probe molecule with all of the required labels. Alternatively, pools of detection probes of a given type can each be labeled with one of the required labels. By combining the pools, the detection probes will, as a group, contain the combination of labels required for that type of detection probe. Where each detection probe is labeled with a single label, label combinations can also be generated by using ATCs with coded combinations of detection tags complementary to the different detection probes. In this scheme, the ATCs will contain a combination of detection tags representing the combination of labels required for a specific label code. Further illustrations are described in U.S. Pat. No. 6,143,495.

Speicher et al. describes a set of fluors and corresponding optical filters spaced across the spectral interval 350-770 nm that give a high degree of discrimination between all possible fluor pairs. This fluor set, which is preferred for combinatorial multicolor coding, consists of 4′-6-diamidino-2-phenylinodole (DAPI), fluorescein (FITC), and the cyanine dyes Cy3, Cy3.5, Cy5, Cy5.5 and Cy7. Any subset of this preferred set can also be used where fewer combinations are required. The absorption and emission maxima, respectively, for these fluors are: DAPI (350 nm; 456 nm), FITC (490 nm; 520 nm), Cy3 (554 nm; 568 nm), Cy3.5 (581 nm; 588 nm), Cy5 (652 nm; 672 nm), Cy5.5 (682 nm; 703 nm) and Cy7 (755 nm; 778 nm). The excitation and emission spectra, extinction coefficients and quantum yield of these fluors are described by Ernst et al., Cytometry 10:3-10 (1989), Mujumdar et al., Cytometry 10:11-19 (1989), Yu, Nucleic Acids Res. 22:3226-3232 (1994), and Waggoner, Meth. Enzymology 246:362-373 (1995). These fluors can all be excited with a 75W Xenon arc.

To attain selectivity, filters with bandwidths in the range of 5 to 16 nm are preferred. To increase signal discrimination, the fluors can be both excited and detected at wavelengths far from their spectral maxima. Emission bandwidths can be made as wide as possible. For low-noise detectors, such as cooled CCD cameras, restricting the excitation bandwidth has little effect on attainable signal to noise ratios. A list of preferred filters for use with the preferred fluor set is listed in Table 1 of Speicher et al. It is important to prevent infra-red light emitted by the arc lamp from reaching the detector; CCD chips are extremely sensitive in this region. For this purpose, appropriate IR blocking filters can be inserted in the image path immediately in front of the CCD window to minimize loss of image quality. Image analysis software can then be used to count and analyze the spectral signatures of fluorescent dots.

5. In Situ Detection Using RCA

In situ detection of nucleic acid molecules is a powerful application of the disclosed method. For example, association of rolling circle replication primers with a nucleic acid (such as a cDNA strand) will associate the resulting TS-DNA with the location of the nucleic acid.

Localization of the TS-DNA for in situ detection can also be enhanced by collapsing the TS-DNA using collapsing detection probes, biotin-antibody conjugates, or both, as described elsewhere herein. Multiplexed in situ detection can be carried out as follows: Rolling circle replication is carried out using unlabeled nucleotides. The different TS-DNAs are then detected using standard multi-color FISH with detection probes specific for each unique detection tag (or other target sequence) in the TS-DNA. Alternatively, and preferably, combinatorial multicolor coding, as described elsewhere herein, can be used for multiplex in situ detection.

6. Enzyme-linked Detection

Amplified nucleic acid labeled by incorporation of labeled nucleotides can be detected with established enzyme-linked detection systems. For example, amplified nucleic acid labeled by incorporation of biotin-16-UTP (Boehringher Mannheim) can be detected as follows. The nucleic acid is immobilized on a solid substrate or support, typically via association of the amplified nucleic acid with an immobilized nucleic acid (such as a cDNA strand) as described elsewhere herein. The substrate is washed and contacted with alkaline phosphatase-streptavidin conjugate (Tropix, Inc., Bedford, Mass.). This enzyme-streptavidin conjugate binds to the biotin moieties on the amplified nucleic acid. The substrate is again washed to remove excess enzyme conjugate and the chemiluminescent substrate CSPD (Tropix, Inc.) is added and covered. The substrate can then be imaged in a Biorad Fluorimager.

7. Collapse of Nucleic Acids

Tandem sequence DNA, which is produced as an extended nucleic acid molecule, can be collapsed into a compact structure. It is preferred that the nucleic acid to be collapsed is immobilized on a substrate. A preferred means of collapsing nucleic acids is by hybridizing one or more collapsing probes with the nucleic acid to be collapsed. Collapsing probes are oligonucleotides having a plurality of portions each complementary to sequences in the nucleic acid to be collapsed. These portions are referred to as complementary portions of the collapsing probe, where each complementary portion is complementary to a sequence in the nucleic acid to be collapsed. The sequences in the nucleic acid to be collapsed are referred to as collapsing target sequences. The complementary portion of a collapsing probe can be any length that supports specific and stable hybridization between the collapsing probe and the collapsing target sequence. For this purpose, a length of 10 to 35 nucleotides is preferred, with a complementary portion of a collapsing probe 16 to 20 nucleotides long being most preferred. It is preferred that at least two of the complementary portions of a collapsing probe be complementary to collapsing target sequences which are separated on the nucleic acid to be collapsed or to collapsing target sequences present in separate nucleic acid molecules. This allows each detection probe to hybridize to at least two separate collapsing target sequences in the nucleic acid sample. In this way, the collapsing probe forms a bridge between different parts of the nucleic acid to be collapsed. The combined action of numerous collapsing probes hybridizing to the nucleic acid will be to form a collapsed network of cross-linked nucleic acid. Collapsed nucleic acid occupies a much smaller volume than free, extended nucleic acid, and includes whatever detection probe or detection label hybridized to the nucleic acid. This result is a compact and discrete nucleic acid structure which can be more easily detected than extended nucleic acid. Collapsing nucleic acids is useful both for in situ hybridization applications and for multiplex detection because it allows detectable signals to be spatially separate even when closely packed. Collapsing nucleic acids is especially preferred for use with combinatorial multicolor coding. Collapsing probes can also contain any of the detection labels described elsewhere herein. TS-DNA collapse can also be accomplished through the use of ligand/ligand binding pairs (such as biotin and avidin) or hapten/antibody pairs. Nucleic acid collapse is further described in U.S. Pat. No. 6,143,495.

F. Processing

There are a number of steps, which can be performed in addition to the basic steps of the disclosed method. These steps can aid in increasing specificity of interactions or strength of the signal or efficiency of replication. The description of these additional steps is not meant to be limiting, but merely illustrative of the adaptability of the disclosed methods.

1. DNA Strand Displacement

DNA strand displacement is one way to amplify TS-DNA. Secondary DNA strand displacement is accomplished by hybridizing secondary DNA strand displacement primers to TS-DNA and allowing a DNA polymerase to synthesize DNA from these primed sites. Because a complement of the secondary DNA strand displacement primer occurs in each repeat of the TS-DNA, secondary DNA strand displacement can result in a high level of amplification. The product of secondary DNA strand displacement is referred to as secondary tandem sequence DNA or TS-DNA-2. Secondary DNA strand displacement can be accomplished by performing RCA to produce TS-DNA, mixing secondary DNA strand displacement primer with the TS-DNA, and incubating under conditions promoting replication of the tandem sequence DNA. The disclosed hairpin open circle probes are especially useful for DNA strand displacement because inactivated hairpin open circle probes will not compete with secondary DNA strand displacement primers for hybridization to TS-DNA. The DNA strand displacement primers are preferably hairpin DNA strand displacement primers.

Secondary DNA strand displacement can also be carried out simultaneously with rolling circle replication. This is accomplished by mixing secondary DNA strand displacement primer with the reaction prior to rolling circle replication. As a secondary DNA strand displacement primer is elongated, the DNA polymerase will run into the 5′ end of the next hybridized secondary DNA strand displacement molecule and will displace its 5′ end. In this fashion a tandem queue of elongating DNA polymerases is formed on the TS-DNA template. As long as the rolling circle reaction continues, new secondary DNA strand displacement primers and new DNA polymerases are added to TS-DNA at the growing end of the rolling circle. For simultaneous rolling circle replication and secondary DNA strand displacement, it is preferred that the rolling circle DNA polymerase be used for both replications. This allows optimum conditions to be used and results in displacement of other strands being synthesized downstream. Secondary DNA strand displacement can follow any DNA replication operation.

Generally, secondary DNA strand displacement can be performed by, simultaneous with or following RCA, mixing a secondary DNA strand displacement primer with the reaction mixture and incubating under conditions that promote both hybridization between the tandem sequence DNA and the secondary DNA strand displacement primer, and replication of the tandem sequence DNA, where replication of the tandem sequence DNA results in the formation of secondary tandem sequence DNA.

When secondary DNA strand displacement is carried out in the presence of a tertiary DNA strand displacement primer, an exponential amplification of TS-DNA sequences takes place. This special and preferred mode of DNA strand displacement is referred to as strand displacement cascade amplification (SDCA). In SDCA, a secondary DNA strand displacement primer primes replication of TS-DNA to form TS-DNA-2, as described elsewhere herein. The tertiary DNA strand displacement primer strand can then hybridize to, and prime replication of, TS-DNA-2 to form TS-DNA-3. Strand displacement of TS-DNA-3 by the adjacent, growing TS-DNA-3 strands makes TS-DNA-3 available for hybridization with secondary DNA strand displacement primer. This results in another round of replication resulting in TS-DNA-4 (which is equivalent to TS-DNA-2). TS-DNA-4, in turn, becomes a template for DNA replication primed by tertiary DNA strand displacement primer. The cascade continues this manner until the reaction stops or reagents become limiting. This reaction amplifies DNA at an almost exponential rate. In a preferred mode of SDCA, the rolling circle replication primer serves as the tertiary DNA strand displacement primer, thus eliminating the need for a separate primer. Optimization of primer concentrations are described in U.S. Pat. No. 6,143,495 and can be aided by analysis of hybridization kinetics (Young and Anderson, “Quantitative analysis of solution hybridization” in Nucleic Acid Hybridization: A Practical Approach (IRL Press, 1985) pages 47-71).

Generally, strand displacement cascade amplification can be performed by, simultaneous with, or following, RCA, mixing a secondary DNA strand displacement primer and a tertiary DNA strand displacement primer with the reaction mixture and incubating under conditions that promote hybridization between the tandem sequence DNA and the secondary DNA strand displacement primer, replication of the tandem sequence DNA—where replication of the tandem sequence DNA results in the formation of secondary tandem sequence DNA—hybridization between the secondary tandem sequence DNA and the tertiary DNA strand displacement primer, and replication of secondary tandem sequence DNA—where replication of the secondary tandem sequence DNA results in formation of tertiary tandem sequence DNA (TS-DNA-3).

Secondary and tertiary DNA strand displacement can also be carried out sequentially. Following a first round of secondary DNA strand displacement, a tertiary DNA strand displacement primer can be mixed with the secondary tandem sequence DNA and incubated under conditions that promote hybridization between the secondary tandem sequence DNA and the tertiary DNA strand displacement primer, and replication of secondary tandem sequence DNA, where replication of the secondary tandem sequence DNA results in formation of tertiary tandem sequence DNA (TS-DNA-3). This round of strand displacement replication can be referred to as tertiary DNA strand displacement.

All rounds of strand displacement replication following rolling circle replication can also be referred to collectively as DNA strand displacement or secondary DNA strand displacement. Other forms of secondary DNA strand displacement are described in U.S. Pat. No. 6,143,495.

The DNA generated by DNA strand displacement can be labeled and/or detected using the same labels, labeling methods, and detection methods described for use with TS-DNA. Most of these labels and methods are adaptable for use with nucleic acids in general. A preferred method of labeling the DNA is by incorporation of labeled nucleotides during synthesis.

2. Transcription Following RCA

Once TS-DNA is generated using RCA, further amplification can be accomplished by transcribing the TS-DNA from promoters embedded in the TS-DNA. This combined process, referred to as rolling circle replication with transcription (RCT), or ligation mediated rolling circle replication with transcription (LM-RCT), requires that the OCP or ATC from which the TS-DNA is made have a promoter portion in its spacer region. The promoter portion is then amplified along with the rest of the OCP or ATC resulting in a promoter embedded in each tandem repeat of the TS-DNA. Because transcription, like rolling circle amplification, is a process that can go on continuously (with re-initiation), multiple transcripts can be produced from each of the multiple promoters present in the TS-DNA. RCT effectively adds another level of amplification of ligated OCP sequences. RCT is further described in U.S. Pat. No. 6,143,495.

The transcripts generated in RCT can be labeled and/or detected using the same labels, labeling methods, and detection methods described for use with TS-DNA. Most of these labels and methods are adaptable for use with nucleic acids in general. A preferred method of labeling RCT transcripts is by direct labeling of the transcripts by incorporation of labeled nucleotides, most preferably biotinylated nucleotides, during transcription.

3. ATC Formation

Amplification target circles for use in the disclosed methods are preferably pre-formed. Alternatively, the amplification target circle can be formed as part of the method, for example, by target-mediated ligation of an open circle probe. The ATC can be formed in any process which capable of forming a circular DNA molecule. Typically this process involves hybridization of a 5′-end and the 3′-end of a first linear DNA molecule (generally an open circle probe) to a second DNA molecule such that the 5′- and 3′ ends of the first DNA molecule are juxtaposed to each other and can be ligated in any ligation reaction.

In target-mediated ligation, an open circle probe, optionally in the presence of one or more gap oligonucleotides, is incubated with a target sequence, under suitable hybridization conditions, and then ligated to form a covalently closed circle. The target sequence can be a rolling circle replication primer. The ligated open circle probe is a form of amplification target circle. This operation is similar to ligation of padlock probes described by Nilsson et al., Science, 265:2085-2088 (1994). The ligation operation allows subsequent amplification to be dependent on the presence of a target sequence. Suitable ligases for the ligation operation are described elsewhere herein. Ligation conditions are generally known. Most ligases require Mg⁺⁺. There are two main types of ligases, those that are ATP-dependent and those that are NAD-dependent. ATP or NAD, depending on the type of ligase, should be present during ligation.

These ligation methods can be performed particularly well with hairpin open circle probes. Hairpin open circle probes are disclosed in co-pending U.S. patent application Ser. No. 09/803,713 filed on Mar. 9, 2001, which is herein incorporated by reference in its entirety for at least the disclosure related to hairpin probes, there uses and modifications.

The target sequence for an open circle probe can be any nucleic acid or other compound to which the target probe portions of the open circle probe can hybridize in the proper alignment. Target sequences can be found in any nucleic acid molecule from any nucleic acid sample. Thus, target sequences can be in nucleic acids in cell or tissue samples, reactions, and assays. The target sequence can be a rolling circle replication primer. Target sequences can also be artificial nucleic acids (or other compounds to which the target probe portions of the open circle probe can hybridize in the proper alignment).

4. Discrimination between Closely Related Sequences

Specially designed capture probes can be used to discriminate between closely related sequences. Capture probes are designed to hybridize with a particular sequence. The specificity of this hybridization step can be increased by requiring a ligation step which is related to a very particular sequence, such as a point mutation in a gene. In general this type of discrimination is achieved by mixing a capture probe and sub-probe with the nucleic acid sample at a temperature and salt concentration which allows hybridization and performing a ligation reaction to join the capture probe to the sub-probe. If the sequence at the ligation junction is complementary then ligation will efficiently take place, but if the sequence at the junction is less than complementary then ligation at the junction will not take place or will take place at an extremely low level. Thus, nucleic acids in the sample having the particular sequence which produces complementary junction sites will become ligated and the those that do not form such sites will not be ligated. Following the ligation step, the conditions of hybridization can be changed, for example by raising the temperature of hybridization or lowering the salt concentration to conditions in which the unligated capture probes and/or sub-probes will not be able to hybridize, but the ligated capture probe/sub-probes will remain hybridized.

Capture probes and sub-probes can be designed to discriminate between closely related target sequences, such as genetic alleles. Where closely related target sequences differ at a single nucleotide, it is preferred that capture probes and sub-probes be designed with the complement of this nucleotide occurring at one end of the probe.

Ligation of capture probes and sub-probes with a mismatch at the terminus is extremely unlikely because of the combined effects of hybrid instability and enzyme discrimination. Features of capture probes and sub-probes that increase the target-dependency of ligation are generally analogous to such features developed for use with the ligation chain reaction. These features can be incorporated into capture probes and sub-probes in the disclosed methods. In particular, European Patent Application EP0439182 describes several features for enhancing target-dependency in LCR that can be adapted for use in the disclosed methods. In general, only one of the probes in a capture probe/sub-probe pair will be designed to have a terminal mismatch, although both probes can have a terminal mismatch.

A preferred form of sequence discrimination can be accomplished by employing two types of sub-probes. In one embodiment, a single capture probe is used which is the same for both sequences to be hybridized, that is, the capture probe is complementary to both sequences. In this case, two sub-probes, one for each sequence can then be used. In a preferred embodiment, a sub-probe ligation operation can be used. Sequence discrimination would occur by virtue of mutually exclusive ligation events, or extension-ligation events, for which only one of the two sub-probes is competent. Preferably, the discriminator nucleotide would be located at the penultimate nucleotide from the 3′ end of each of the sub-probes.

This same type of increased discrimination through ligation can be utilized for rolling circle replication primers as for capture probes. For example, a full RCRP can be composed of two (or more) parts all of which hybridize with the ATC. After mixing of the RCRP with the manipulated nucleic acid sample, the RCRP can be incubated with the ATC. At this point, additional parts of the full RCRP designed to be ligated on to the 3′ end of the RCRP through hybridization on the ATC template can be added. Once the RCRP primer is completed, that is the parts are ligated together, the stringency of the hybridization can be increased until only full ATC:RCRP complexes remain.

Likewise, any type of nucleic acid hybridization step to be use in or with the disclosed method can be modified in this way to increase the specificity of the ultimate product.

5. Gap-Filling Ligation

When the OCP hybridizes to the target sequence the OCP can either be hybridized such that the 5′ and 3′ ends are immediately juxtaposed or such that there is a gap between the two ends. The gap space formed by an OCP hybridized to a target sequence is normally occupied by one or more gap oligonucleotides as described herein. Such a gap space may also be filled in by a gap-filling DNA polymerase during the ligation operation. As an alternative, the gap space can be partially bridged by one or more gap oligonucleotides, with the remainder of the gap filled using DNA polymerase. This modified ligation operation is referred to herein as gap-filling ligation and is a preferred form of the ligation operation. The principles and procedure for gap-filling ligation are generally analogous to the filling and ligation performed in gap LCR (Wiedmann et al., PCR Methods and Applications (Cold Spring Harbor Laboratory Press, Cold Spring Harbor Laboratory, NY, 1994) pages S51-S64; Abravaya et al., Nucleic Acids Res., 23(4):675-682 (1995); European Patent Application EP0439182 (1991)). In the case of target-mediated ligation, the gap-filling ligation operation is substituted for the normal ligation operation. Gap-filling ligation provides a means for discriminating between closely related target sequences. Gap-filling ligation can be accomplished by using a different DNA polymerase, referred to herein as a gap-filling DNA polymerase. Suitable gap-filling DNA polymerases are described elsewhere herein. Alternatively, DNA polymerases in general can be used to fill the gap when a stop base is used. The use of stop bases in the gap-filling operation of LCR is described in European Patent Application EP0439182. The principles of the design of gaps and the ends of flanking probes to be joined, as described in EP0439182, is generally applicable to the design of the gap spaces and the ends of target probe portions described herein. Gap-filling ligation is further described in U.S. Pat. No. 6,143,495.

6. Size Classes of Tandem Sequence DNA

Rolling circle amplification can be engineered to produce TS-DNA of different lengths in an assay involving multiple ligated OCPs or ATCs. This can be useful for extending the number of different molecules that can be detected in a single assay. Techniques for producing size classes of TS-DNA are described in U.S. Pat. No. 6,143,495.

7. Particular Embodiments

There are many different variations of the disclosed methods some of which are discussed elsewhere herein. For example, disclosed is a method of amplifying messenger RNA, the method comprising (a) mixing one or more RT primers with a nucleic acid sample and reverse transcribing to produce cDNA strands each comprising one of the RT primers, (b) fragmenting the cDNA strands to form fragmented cDNA, (c) adding a capture tag to the fragmented cDNA, (d) mixing the fragmented cDNA with a set of capture probes under conditions that promote hybridization of the fragmented cDNA to the capture probes, (e) mixing one or more rolling circle replication primers with the fragmented cDNA under conditions that promote association of the fragmented cDNA with the rolling circle replication primers, and wherein the association occurs via the capture tag, (f) mixing one or more amplification target circles with the rolling circle replication primers under conditions that promote association of the rolling circle replication primers with the amplification target circles, (g) incubating the amplification target circles under conditions that promote replication of the amplification target circles, wherein replication of the amplification target circles results in the formation of tandem sequence DNA.

In some variations of this embodiment (as well as others) the rolling circle replication primers each comprise a capture tag. In other embodiments of the association of the rolling circle replication primers with the cDNA occurs via association of the capture tag added to the fragmented cDNA and the capture tag in the rolling circle replication primers.

In certain disclosed methods the capture tag is added to the fragmented cDNA by terminal transferase or for example the capture tag is biotinylated-ddNTP.

Another disclosed method is a method of amplifying messenger RNA, the method comprising (a) mixing one or more RT primers with a nucleic acid sample and reverse transcribing to produce cDNA strands each comprising one of the RT primers, wherein each RT primer comprises a reverse transcription primer portion and a capture tag, (b) mixing the cDNA strands with a set of capture probes under conditions that promote hybridization of the cDNA strands to the capture probes, (c) mixing one or more rolling circle replication primers with the cDNA strands under conditions that promote association of the cDNA strands to the rolling circle replication primers, and wherein the association occurs through the capture tag, (d) mixing one or more amplification target circles with the rolling circle replication primers under conditions that promote association of the rolling circle replication primers with the amplification target circles, (e) incubating the amplification target circles under conditions that promote replication of the amplification target circles, wherein replication of the amplification target circles results in the formation of tandem sequence DNA.

For example, it is understood that methods where the rolling circle replication primers each comprise a capture tag or where association of the rolling circle replication primers with the cDNA occurs via association of the capture tag added to the cDNA and the capture tag in the rolling circle replication primers are disclosed.

Other embodiments disclosed are methods of amplifying messenger RNA, the method comprising (a) mixing one or more RT primers with a nucleic acid sample and reverse transcribing to produce cDNA strands each comprising one of the RT primers, wherein each RT primer comprises a reverse transcription primer portion, wherein the cDNA comprises a capture tag, (b) mixing the cDNA strands with a set of capture probes under conditions that promote hybridization of the cDNA strands to the capture probes, (c) mixing one or more rolling circle replication primers with the cDNA strands under conditions that promote association of the cDNA strands with the rolling circle replication primers, and wherein the association occurs through the capture tag, (d) mixing one or more amplification target circles with the rolling circle replication primers under conditions that promote association of the rolling circle replication primers with the amplification target circles, (e) incubating the amplification target circles under conditions that promote replication of the amplification target circles, wherein replication of the amplification target circles results in the formation of tandem sequence DNA.

Other methods include rolling circle replication primers where each comprise a capture tag, or wherein the association of the rolling circle replication primers with the cDNA occurs via association of the capture tag incorporated into the cDNA and the capture tag in the rolling circle replication primers, or where the capture tag is derived from allyl amine dUTP.

In certain embodiments the amplification target circle hybridizes with a rolling circle amplification primer comprising an NHS ester or where the capture tag is derived from incorporation of biotinylated-ddNTP into the cDNA.

A further embodiment is a method of amplifying messenger RNA, the method comprising (a) mixing one or more RT primers with a nucleic acid sample and reverse transcribing to produce cDNA strands each comprising one of the RT primers, wherein each RT primer comprises a reverse transcription primer portion and a rolling circle replication primer portion, wherein the reverse transcription primer portion and the rolling circle replication primer portion each comprise a 5′ end, wherein the reverse transcription primer portion and the rolling circle replication primer portion are not linked via their 5′ ends, (b) mixing the cDNA strands with a set of capture probes under conditions that promote hybridization of the cDNA strands to the capture probes, (c) mixing one or more amplification target circles with the rolling circle replication primer portions under conditions that promote association of the rolling circle replication primer portions with the amplification target circles, (d) incubating the amplification target circles under conditions that promote replication of the amplification target circles, wherein replication of the amplification target circles results in the formation of tandem sequence DNA.

Disclosed is method of using messenger RNA, the method comprising replicating one or more amplification target circles to produce one or more tandem sequence DNAs, wherein each tandem sequence DNA is coupled to a rolling circle replication primer, wherein the rolling circle replication primer is associated with a cDNA strand, wherein the rolling circle replication primer comprises a capture tag, wherein the association occurs via the capture tag, wherein the cDNA strand is hybridized to a capture probe, wherein the cDNA strand comprises an RT primer, wherein the cDNA strand is produced by reverse transcribing a nucleic acid sample with the RT primer.

Also disclosed is a method of using messenger RNA, the method comprising replicating one or more amplification target circles to produce one or more tandem sequence DNAs, wherein each tandem sequence DNA is coupled to a rolling circle replication primer, wherein the rolling circle replication primer is associated with a fragmented cDNA strand, wherein the fragmented cDNA strand is hybridized to a capture probe, wherein the fragmented cDNA comprises a capture tag, wherein the association occurs via the capture tag, wherein the fragmented cDNA strand is a fragment of a cDNA strand, wherein the cDNA strand comprises an RT primer, wherein the cDNA strand is produced by reverse transcribing a nucleic acid sample with the RT primer.

In addition, disclosed is a method of using messenger RNA, the method comprising replicating one or more amplification target circles to produce one or more tandem sequence DNAs, wherein each tandem sequence DNA is coupled to a rolling circle replication primer, wherein the rolling circle replication primer is associated with a cDNA strand, wherein the cDNA strand is hybridized to a capture probe, wherein the cDNA strand comprises an RT primer, wherein the RT primer comprises a capture tag, wherein the association occurs via the capture tag, wherein the cDNA strand is produced by reverse transcribing a nucleic acid sample with the RT primer.

Other disclosed methods include a method of using messenger RNA, the method comprising replicating one or more amplification target circles to produce one or more tandem sequence DNAs, wherein each tandem sequence DNA is coupled to a rolling circle replication primer, wherein the rolling circle replication primer is associated with a cDNA strand, wherein the cDNA strand comprises a capture tag, wherein the association occurs via the capture tag, wherein the cDNA strand is hybridized to a capture probe, wherein the cDNA strand comprises an RT primer, wherein the cDNA strand is produced by reverse transcribing a nucleic acid sample with the RT primer.

Disclosed is a method of using messenger RNA, the method comprising replicating one or more amplification target circles to produce one or more tandem sequence DNAs, wherein each tandem sequence DNA is coupled to a rolling circle replication primer portion of an RT primer that comprises the rolling circle replication primer portion and a reverse transcription primer portion, wherein the cDNA strand is hybridized to a capture probe, wherein the cDNA strand comprises the RT primer, wherein the cDNA strand is produced by reverse transcribing a nucleic acid sample with the RT primer, wherein the reverse transcription primer portion and the rolling circle replication primer portion each comprise a 5′ end, wherein the reverse transcription primer portion and the rolling circle replication primer portion are not linked via their 5′ ends.

### Compositions

The disclosed methods and compositions can include a number of different parts or materials. In many embodiments, these parts may or may not be involved in a particular embodiment of a disclosed method and composition. This section, while not intended o be limiting, addresses some of the variations on the materials that can be used in the disclosed methods and compositions. For example, discussed below are nucleic acids, RT primers, nucleic acid samples, cDNA strands, capture probes, rolling circle replication primer, capture tag systems, amplification target circles, tandem sequence DNA, open circle probes, gap oligonucleotides, DNA strand displacement primers, reporter binding agents, detection labels, detection probes, address probes, oligonucleotide synthesis, solid state detectors, DNA ligases, DNA polymerases, RNA polymerases, and various kits. Each of these is either used in the disclosed method or is used in a variation of the disclosed method. Examples of how use these various materials can be found through out the specification.

A. RT Primers

RT primers are used to prime reverse transcription to form cDNA strands. RT primers can be made up of any nucleotide, nucleotide analog, nucleotide substitute, or nucleotide conjugate, as long as the RT primer is capable of priming reverse transcription. In certain embodiments the RT primer is attached to a capture tag. This capture tag can be used for a variety of manipulations, including interactions with another capture tag attached to the rolling circle amplification primer.

RT primers have sequence complementary to a primer complement portion of a mRNA. This sequence is referred to as the complementary portion of the RT primer. The complementary portion of an RT primer can be any sequence, including a poly T sequence designed to interact with the poly A tail of mRNA. The RT primer can also include sequence that is specific to a target mRNA so that the subsequence cDNA is a unique cDNA or subset of all the possible cDNAs which could be generated from the mRNA library. The specific sequence can either be at the 5′ end of the mRNA and be juxtaposed to the poly A tail or the specific sequence can be anywhere within the mRNA sequence. A specific RT sequence can be attached to a poly T sequence in certain embodiments.

The RT primer can be any size desired. The conditions of the RT reaction can be varied to efficiently utilize different sized RT primers. The complementary portion of a RT primer can be any length that supports specific and stable hybridization between the primer and the target mRNA. Generally this is 10 to 35 nucleotides long, but is preferably 16 to 20 nucleotides long.

The RT primer can be linked to the rolling circle replication primer. In some embodiments, the RT primer is not linked to an RCRP via a 5′-5′ phosphodiester bond. In other embodiments the RT primer is not linked to an RCRP via any 5′-5′ bond. In still other embodiments, the RT-primer is not linked covalently to the RCRP.

B. Nucleic Acid Samples

The nucleic acid sample can be derived from any source that has, or is suspected of having, nucleic acids. A nucleic acid sample the source of nucleic acids upon which a manipulation, such as reverse transcription, transcription or DNA replication, is performed. The nucleic acid sample will typically contain a target nucleic acid, for example a specific mRNA or pool of mRNA molecules. The nucleic acid sample can contain RNA or DNA or both. The nucleic acid sample in certain embodiments can also include chemically synthesized nucleic acids. The nucleic acid sample can include any nucleotide, nucleotide analog, nucleotide substitute or nucleotide conjugate.

C. cDNA Strands

The cDNA strands are nucleic acid molecules that are derived from the manipulation of mRNA, specifically through reverse transcription. In certain embodiments, however, the cDNA simply represents a complement copy of the cognate mRNA sequence. The cDNA strands can possess any nucleotide, nucleotide analog, nucleotide substitute, or nucleotide conjugate that can be enzymatically incorporated or made by post reverse transcription modification.

D. Capture Probes

A capture probe is an oligonucleotide having sequence complementary to a sequence in a base nucleic acid or in a manipulated product nucleic acid. This sequence is referred to as the complementary portion of the capture probe. The complementary portion of a capture probe generally will be complementary to a specific sequence in a target nucleic acid molecule. The complementary portion of a capture probe can be any length that supports specific and stable hybridization between the probe and the target nucleic acid. Generally this is 10 to 35 nucleotides long, but is preferably 16 to 20 nucleotides long.

The capture probes typically can be attached to a substrate as discussed elsewhere herein. The capture probes can contain any nucleotide, nucleotide analog, nucleotide substitute, or nucleotide conjugate. The capture probes are designed to interact, typically through hybridization, with other nucleic acids, typically contained within the nucleic acid sample or in the manipulated nucleic acid sample. In certain embodiments of the disclosed methods and compositions the capture probes can comprise a capture tag.

E. Solid-State Detectors

Solid-state detectors are solid-state substrates or supports to which capture probes have been coupled. A preferred form of solid-state detector is an array detector. An array detector is a solid-state detector to which multiple different capture probes have been coupled in an array, grid, or other organized pattern.

Solid-state substrates for use in solid-state detectors can include any solid material to which oligonucleotides can be coupled. This includes materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Solid-state substrates can have any useful form including thin films or membranes, beads, bottles, dishes, fibers, optical fibers, woven fibers, shaped polymers, particles and microparticles. A preferred form for a solid-state substrate is a microtiter dish.

Capture probes immobilized on a solid-state substrate allow capture of nucleic acids on a solid-state detector. Such capture provides a convenient means of washing away reaction components that might interfere with subsequent detection steps. By attaching different capture probes to different regions of a solid-state detector, different nucleic acids can be captured at different, and therefore diagnostic, locations on the solid-state detector.

Methods for immobilization of oligonucleotides to solid-state substrates are well established. Oligonucleotides, such as capture probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A preferred method of attaching oligonucleotides to solid-state substrates is described by Guo et al., Nucleic Acids Res. 22:5456-5465 (1994).

F. Rolling Circle Replication Primers

A rolling circle replication primer (RCRP) is an oligonucleotide having sequence complementary to the primer complement portion of an OCP or ATC. This sequence is referred to as the complementary portion of the RCRP. The complementary portion of a RCRP and the cognate primer complement portion can have any desired sequence so long as they are complementary to each other. In general, the sequence of the RCRP can be chosen such that it is not significantly complementary to any other portion of the OCP or ATC. The complementary portion of a rolling circle replication primer can be any length that supports specific and stable hybridization between the primer and the primer complement portion. Generally this is 10 to 35 nucleotides long, but is preferably 16 to 20 nucleotides long.

Preferred rolling circle replication primers for use in the disclosed method can form an intramolecular stem structure involving one or both of the RCRP's ends. Such rolling circle replication primers are referred to herein as hairpin rolling circle replication primers. An intramolecular stem structure involving an end refers to a stem structure where the terminal nucleotides (that is, nucleotides at the end) of the RCRP are hybridized to other nucleotides in the RCRP.

It is preferred that rolling circle replication primers also contain additional sequence at the 5′ end of the RCRP that is not complementary to any part of the OCP or ATC. This sequence is referred to as the non-complementary portion of the RCRP. The non-complementary portion of the RCRP, if present, serves to facilitate strand displacement during DNA replication. The non-complementary portion of a RCRP may be any length, but is generally 1 to 100 nucleotides long, and preferably 4 to 8 nucleotides long. A rolling circle replication primer can be used as the tertiary DNA strand displacement primer in strand displacement cascade amplification.

Rolling circle replication primers may also include modified nucleotides to make them resistant to exonuclease digestion. For example, the primer can have three or four phosphorothioate linkages between nucleotides at the 5′ end of the primer. Such nuclease resistant primers allow selective degradation of excess unligated OCP and gap oligonucleotides that might otherwise interfere with hybridization of detection probes, address probes, and secondary OCPs to the amplified nucleic acid. RCRPs may in certain embodiments comprise capture tags.

G. Capture Tags

Capture tags can be used to associate molecules which have a capture tag with other molecules. Capture tags can also be used to separate molecules having a capture tag away from molecules which do not. As used herein, a capture tag is any compound that can be attached either covalently or non-covalently with a molecule of choice including a nucleic acid molecule or a protein molecule, and which can be used to at least separate, identify, associate, denote, or mark compounds or complexes having the capture tag from those that do not.

Preferably, a capture tag is a compound, such as a ligand or hapten, that binds to or interacts with another compound, such as a ligand-binding molecule or an antibody. It is also preferred that such interaction between the capture tag and the molecule that interacts with the capture tag be a specific interaction, such as between a hapten and an antibody or a ligand and a ligand-binding molecule. Thus, capture tags are molecules which can function as a ligand or as a receptor for a ligand. Thus, a capture tag could be either the hapten or the antibody that binds the hapten. Therefore, in a preferred embodiment, two capture tags will interact specifically with each other. When two capture tags specifically interact with each other this is called a capture tag pair.

Suitable capture tags include hapten or ligand molecules that can be coupled to the 5′ end of the synthesized RNA molecule. Preferred capture tags, described in the context of nucleic acid probes, have been described by Syvanen et al., Nucleic Acids Res., 14:5037 (1986)). Preferred capture tags include biotin, which can be incorporated into nucleic acids (Langer et al., Proc. Natl. Acad. Sci. USA 78:6633 (1981)) and captured using streptavadin or biotin-specific antibodies. A preferred hapten for use as a capture tag is digoxygenin (Kerkhof, Anal. Biochem. 205:359-364 (1992)). Many compounds for which a specific antibody is known or for which a specific antibody can be generated can be used as capture tags. Such capture tags can be captured by antibodies which recognize the compound. Antibodies useful as capture tags can be obtained commercially or produced using well established methods. For example, Johnstone and Thorpe, Immunochemistry In Practice (Blackwell Scientific Publications, Oxford, England, 1987), on pages 30-85, describe general methods useful for producing both polyclonal and monoclonal antibodies.

Another preferred capture tag is an anti-antibody capture tag, which can form a capture tag pair between the anti-antibody and its cognate antibody. Such anti-antibody antibodies and their use are well known. For example, anti-antibody antibodies that are specific for antibodies of a certain class (for example, IgG, IgM), or antibodies of a certain species (for example, anti-rabbit antibodies) are commonly used to detect or bind other groups of antibodies. Thus, one can have an antibody one capture tag and then this antibody:capture tag complex can then be purified by binding to another antibody for the antibody portion of the complex.

Another type of capture tag is one which can form selectable cleavable covalent bonds with other molecules of choice. For example, a preferred capture tag of this type is one which contains a sulfur atom. An RNA molecule which is associated with this capture tag can be purified by retention on a thiolpropyl sepharose column. Extensive washing of the column removes unwanted molecules and reduction with β-mercaptoethanol, for example, allows the desired RNA molecules to be collected after purification under relatively gentle conditions (See Lorsch and Szostak, 1994 for a reduction to practice of this type of capture tag which is herein incorporated by reference).

Capture tags can be for example, biotin, digoxigenin, bromodeoxyuridine, or other hapten. A capture tag could also be for example, biotinylated-ddNTP, or just biotin. A capture tag could also be for example, allyl amine dUTP, or just allyl amine.

Capture tags and capture tags pairs can be built around, vitamins, such as biotin, (Langer et al., PNAS USA, 78:6633), haptens such as digoxigenin (Kessler, Mol. Cell. Probes, 5:161 (1991), fluorescein (Holtke et al., Anal. Biochem., 207:24 (1992), dinitrophenyl (Lichter, et al., Science 247:64 (1990), bromodeoxyuridine (Porstmann et al., J. Immunol. Meth. 82:169 (1985)), sulfone Nur et al., Non radioactive labeling and detection of biomolecules pp: 110-115 (1992)) Springer-verlag), or immunogold (Hayat et al. Ed., Colloidal Gold, principles, methods and applications Vols 1 and 2 Academic Press, N.Y. (1989)) each of which is herein specifically incorporated by reference. Heavy metal systems can also be used.

There are a number of capture tags, capture tag systems or capture tag pairs which are commercially available.

H. Amplification Target Circles

An amplification target circle (ATC) is a circular single-stranded DNA molecule, preferably containing between 40 to 1000 nucleotides, more preferably between about 50 to 150 nucleotides, and most preferably between about 50 to 100 nucleotides. Portions of ATCs have specific functions making the ATC useful for rolling circle amplification (RCA). These portions are referred to as the primer complement portion, the detection tag portions, the secondary target sequence portions, the address tag portions, and the promoter portion. The primer complement portion is a required element of an amplification target circle. Detection tag portions, secondary target sequence portions, address tag portions, and promoter portions are optional. The primer complement portion, and the detection tag portions, the secondary target sequence portions, the address tag portions, and the promoter portion, if present, are preferably non-overlapping. However, various of these portions can be partially or completely overlapping if desired. Generally, an amplification target circle is a single-stranded, circular DNA molecule comprising a primer complement portion. Those segments of the ATC that do not correspond to a specific portion of the ATC can be arbitrarily chosen sequences. It is preferred that ATCs do not have any sequences that are self-complementary. It is considered that this condition is met if there are no complementary regions greater than six nucleotides long without a mismatch or gap. It is also preferred that ATCs containing a promoter portion do not have any sequences that resemble a transcription terminator, such as a run of eight or more thymidine nucleotides. Ligated open circle probes are a type of ATC, and as used herein the term amplification target circle includes ligated open circle probes. An ATC can be used in the same manner as described herein for OCPs that have been ligated.

1. Primer Complement Portion

The primer complement portion of an ATC is complementary to the rolling circle replication primer (RCRP). Each ATC preferably has a single primer complement portion. This allows rolling circle replication to initiate at a single site on ligated ATCs. The primer complement portion and the cognate primer can have any desired sequence so long as they are complementary to each other. The sequence of the primer complement portion is referred to as the primer complement sequence. In general, the sequence of the primer complement can be chosen such that it is not significantly similar to any other portion of the ATC. The primer complement portion can be any length that supports specific and stable hybridization between the primer complement portion and the primer. For this purpose, a length of 10 to 35 nucleotides is preferred, with a primer complement portion 16 to 20 nucleotides long being most preferred. The primer complement portion can be located anywhere within the spacer region of an ATC.

2. Detection Tag Portions

Detection tag portions have sequences matching the sequence of the complementary portion of detection probes. These detection tag portions, when amplified during rolling circle replication, result in TS-DNA having detection tag sequences that are complementary to the complementary portion of detection probes. If present, there may be one, two, three, or more than three detection tag portions on an ATC. It is preferred that an ATC have two, three or four detection tag portions. Most preferably, an ATC will have three detection tag portions. Generally, it is preferred that an ATC have 60 detection tag portions or less. There is no fundamental limit to the number of detection tag portions that can be present on an ATC except the size of the ATC. When there are multiple detection tag portions, they may have the same sequence or they may have different sequences, with each different sequence complementary to a different detection probe. It is preferred that an ATC contain detection tag portions that have the same sequence such that they are all complementary to a single detection probe. For some multiplex detection methods, it is preferable that ATCs contain up to six detection tag portions and that the detection tag portions have different sequences such that each of the detection tag portions is complementary to a different detection probe. The detection tag portions can each be any length that supports specific and stable hybridization between the detection tags and the detection probe. For this purpose, a length of 10 to 35 nucleotides is preferred, with a detection tag portion 15 to 20 nucleotides long being most preferred.

3. Secondary Target Sequence Portions

Secondary target sequence portions have sequences matching the sequence of target probe portions of an open circle probe. These secondary target sequence portions, when amplified during rolling circle replication, result in TS-DNA having secondary target sequences that are complementary to target probe portions of an open circle probe. If present, there may be one, two, or more than two secondary target sequence portions on an ATC. It is preferred that an ATC have one or two secondary target sequence portions. Most preferably, an ATC will have one secondary target sequence portion. Generally, it is preferred that an ATC have 50 secondary target sequence portions or less. There is no fundamental limit to the number of secondary target sequence portions that can be present on an ATC except the size of the ATC. When there are multiple secondary target sequence portions, they may have the same sequence or they may have different sequences, with each different sequence complementary to a different secondary ATC. It is preferred that an ATC contain secondary target sequence portions that have the same sequence such that they are all complementary to a single target probe portion of a secondary ATC. The secondary target sequence portions can each be any length that supports specific and stable hybridization between the secondary target sequence and the target probes of its cognate OCP. For this purpose, a length of 20 to 70 nucleotides is preferred, with a secondary target sequence portion 30 to 40 nucleotides long being most preferred.

4. Address Tag Portion

Address tag portions have sequence matching the sequence of the complementary portion of an address probe. This address tag portion, when amplified during rolling circle replication, results in TS-DNA having address tag sequences that are complementary to the complementary portion of address probes. If present, there may be one, or more than one, address tag portions on an ATC. It is preferred that an ATC have one or two address tag portions. Most preferably, an ATC will have one address tag portion. Generally, it is preferred that an ATC have 50 address tag portions or less. There is no fundamental limit to the number of address tag portions that can be present on an ATC except the size of the ATC. When there are multiple address tag portions, they may have the same sequence or they may have different sequences, with each different sequence complementary to a different address probe. It is preferred that an ATC contain address tag portions that have the same sequence such that they are all complementary to a single address probe. The address tag portion can overlap all or a portion of target probe portions (if present), and all of any intervening gap space (if present). The address tag portion can be any length that supports specific and stable hybridization between the address tag and the address probe. For this purpose, a length between 10 and 35 nucleotides long is preferred, with an address tag portion 15 to 20 nucleotides long being most preferred.

5. Promoter Portion

The promoter portion corresponds to the sequence of an RNA polymerase promoter. A promoter portion can be included in an ATC so that transcripts can be generated from TS-DNA. The sequence of any promoter may be used, but simple promoters for RNA polymerases without complex requirements are preferred. It is also preferred that the promoter is not recognized by any RNA polymerase that may be present in the nucleic acid sample. Preferably, the promoter portion corresponds to the sequence of a T7 or SP6 RNA polymerase promoter. The T7 and SP6 RNA polymerases are highly specific for particular promoter sequences. Other promoter sequences specific for RNA polymerases with this characteristic would also be preferred. Because promoter sequences are generally recognized by specific RNA polymerases, the cognate polymerase for the promoter portion of the ATC should be used for transcriptional amplification. Numerous promoter sequences are known and any promoter specific for a suitable RNA polymerase can be used. The promoter portion can be located anywhere within the spacer region of an ATC and can be in either orientation.

I. Tandem Sequence DNA

An amplification target circle, when replicated, gives rise to a long DNA molecule containing multiple repeats of sequences complementary to the amplification target circle. This long DNA molecule is referred to herein as tandem sequences DNA (TS-DNA). TS-DNA contains sequences complementary to the primer complement portion and, if present on the amplification target circle, the detection tag portions, the secondary target sequence portions, the address tag portions, and the promoter portion. These sequences in the TS-DNA are referred to as primer sequences (which match the sequence of the rolling circle replication primer), spacer sequences (complementary to the spacer region), detection tags, secondary target sequences, address tags, and promoter sequences. Amplification target circles are useful as tags for specific binding molecules.

J. DNA Strand Displacement Primers

Primers used for secondary DNA strand displacement are referred to herein as DNA strand displacement primers. One form of DNA strand displacement primer, referred to herein as a secondary DNA strand displacement primer, is an oligonucleotide having sequence matching part of the sequence of an OCP or ATC. This sequence is referred to as the matching portion of the secondary DNA strand displacement primer. This matching portion of a secondary DNA strand displacement primer is complementary to sequences in TS-DNA. The matching portion of a secondary DNA strand displacement primer may be complementary to any sequence in TS-DNA. However, it is preferred that it not be complementary TS-DNA sequence matching either the rolling circle replication primer or a tertiary DNA strand displacement primer, if one is being used. This prevents hybridization of the primers to each other. The matching portion of a secondary DNA strand displacement primer can be any length that supports specific and stable hybridization between the primer and its complement. Generally this is 12 to 35 nucleotides long, but is preferably 18 to 25 nucleotides long.

Preferred secondary DNA strand displacement primers for use in the disclosed method can form an intramolecular stem structure involving one or both of the secondary DNA strand displacement primer's ends. Such secondary DNA strand displacement primers are referred to herein as hairpin secondary DNA strand displacement primers. An intramolecular stem structure involving an end refers to a stem structure where the terminal nucleotides (that is, nucleotides at the end) of the secondary DNA strand displacement primer are hybridized to other nucleotides in the secondary DNA strand displacement primer. The formation of the intramolecular stem structure during replication allows the structure to reduce or prevent priming by secondary DNA strand displacement primers at unintended sequences. In particular, the intramolecular stem structure prevents the secondary DNA strand displacement primer in which the structure forms from priming nucleic acid replication at sites other than primer complement sequences (that is, the specific sequences complementary to the complementary portion of the secondary DNA strand displacement primer) in TS-DNA. A secondary DNA strand displacement primer that forms a stem and loop structure with a portion of the matching portion in the loop can be designed so that hybridization of the matching portion in the loop to the primer complement sequence disrupts the intramolecular stem structure (Tyagi and Kramer, Nat Biotechnol 14(3):303-8 (1996); Bonnet et al., Proc Natl Acad Sci U S A 96(11):6171-6 (1999)). In this way, the intramolecular stem structure remains intact in the absence of the primer complement sequence and thus reduces or eliminates the ability of the secondary DNA strand displacement primer to prime nucleic acid replication. In the presence of the primer complement sequence, disruption of the intramolecular stem structure allows the end of the secondary DNA strand displacement primer to hybridize to the primer complement sequence.

Another form of DNA strand displacement primer, referred to herein as a tertiary DNA strand displacement primer, is an oligonucleotide having sequence complementary to part of the sequence of an OCP or ATC. This sequence is referred to as the complementary portion of the tertiary DNA strand displacement primer. This complementary portion of the tertiary DNA strand displacement primer matches sequences in TS-DNA. The complementary portion of a tertiary DNA strand displacement primer may be complementary to any sequence in the OCP or ATC. However, it is preferred that it not be complementary OCP or ATC sequence matching the secondary DNA strand displacement primer. This prevents hybridization of the primers to each other. Preferably, the complementary portion of the tertiary DNA strand displacement primer has sequence complementary to a portion of the spacer portion of an OCP. The complementary portion of a tertiary DNA strand displacement primer can be any length that supports specific and stable hybridization between the primer and its complement. Generally this is 12 to 35 nucleotides long, but is preferably 18 to 25 nucleotides long. Preferred tertiary DNA strand displacement primers for use in the disclosed method can form an intramolecular stem structure involving one or both of the tertiary DNA strand displacement primer's ends in the same manner as hairpin secondary DNA strand displacement primers. Such tertiary DNA strand displacement primers are referred to herein as hairpin tertiary DNA strand displacement primers.

Discrimination of DNA strand displacement primer hybridization also can be accomplished by hybridizing primer to primer complement portions in TS-DNA under conditions that favor only exact sequence matches leaving other DNA strand displacement primer unhybridized. The unhybridized DNA strand displacement primers will retain or re-form the intramolecular hybrid and the end of the DNA strand displacement primer involved in the intramolecular stem structure will be extended during replication.

It is preferred that secondary and tertiary DNA strand displacement primers also contain additional sequence at their 5′ end that is not complementary to any part of the OCP or ATC. This sequence is referred to as the non-complementary portion of the secondary or tertiary DNA strand displacement primer. The non-complementary portion of the DNA strand displacement primer, if present, serves to facilitate strand displacement during DNA replication. The non-complementary portion of a DNA strand displacement primer may be any length, but is generally 1 to 100 nucleotides long, and preferably 4 to 8 nucleotides long. A rolling circle replication primer is a preferred form of tertiary DNA strand displacement primer.

DNA strand displacement primers may also include modified nucleotides to make them resistant to exonuclease digestion. For example, the primer can have three or four phosphorothioate linkages between nucleotides at the 5′ end of the primer. Such nuclease resistant primers allow selective degradation of excess unligated OCP and gap oligonucleotides that might otherwise interfere with hybridization of detection probes, address probes, and secondary OCPs to the amplified nucleic acid. DNA strand displacement primers can be used for secondary DNA strand displacement and strand displacement cascade amplification, both described elsewhere herein and in U.S. Pat. No. 6,143,495.

K. Detection Labels

To aid in detection and quantitation of nucleic acids amplified in the disclosed methods, detection labels can be directly incorporated into amplified nucleic acids or can be coupled to detection molecules. As used herein, a detection label is any molecule that can be associated with amplified nucleic acid, directly or indirectly, and which results in a measurable, detectable signal, either directly or indirectly. Many such labels for incorporation into nucleic acids or coupling to nucleic acid or antibody probes are known to those of skill in the art. Examples of detection labels suitable for use in the disclosed method are radioactive isotopes, fluorescent molecules, phosphorescent molecules, enzymes, antibodies, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, OR and Research Organics, Cleveland, Ohio.

Labeled nucleotides are preferred form of detection label since they can be directly incorporated into the products of RCA and RCT during synthesis. Examples of detection labels that can be incorporated into amplified DNA or RNA include nucleotide analogs such as BrdUrd (Hoy and Schimke, Mutation Research 290:217-230 (1993)), BrUTP (Wansick et al., J. Cell Biology 122:283-293 (1993)) and nucleotides modified with biotin (Langer et al., Proc. Natl. Acad. Sci. USA 78:6633 (1981)) or with suitable haptens such as digoxygenin (Kerkhof, Anal. Biochem. 205:359-364 (1992)). Suitable fluorescence-labeled nucleotides are Fluorescein-isothiocyanate-dUTP, Cyanine-3-dUTP and Cyanine-5-dUTP (Yu et al., Nucleic Acids Res., 22:3226-3232 (1994)). A preferred nucleotide analog detection label for DNA is BrdUrd (BUDR triphosphate, Sigma), and a preferred nucleotide analog detection label for RNA is Biotin-16-uridine-5′-triphosphate (Biotin-16-dUTP, Boehringher Mannheim). Fluorescein, Cy3, and Cy5 can be linked to dUTP for direct labeling. Cy3.5 and Cy7 are available as avidin or anti-digoxygenin conjugates for secondary detection of biotin- or digoxygenin-labeled probes.

Detection labels that are incorporated into amplified nucleic acid, such as biotin, can be subsequently detected using sensitive methods well-known in the art. For example, biotin can be detected using streptavidin-alkaline phosphatase conjugate (Tropix, Inc.), which is bound to the biotin and subsequently detected by chemiluminescence of suitable substrates (for example, chemiluminescent substrate CSPD: disodium, 3-(4-methoxyspiro-[1,2,-dioxetane-3-2′-(5′-chloro)tricyclo [3.3.1.1^{3,7}]decane]-4-yl) phenyl phosphate; Tropix, Inc.).

A preferred detection label for use in detection of amplified RNA is acridinium-ester-labeled DNA probe (GenProbe, Inc., as described by Arnold et al., Clinical Chemistry 35:1588-1594 (1989)). An acridinium-ester-labeled detection probe permits the detection of amplified RNA without washing because unhybridized probe can be destroyed with alkali (Arnold et al. (1989)).

Molecules that combine two or more of these detection labels are also considered detection labels. Any of the known detection labels can be used with the disclosed probes, tags, and method to label and detect nucleic acid amplified using the disclosed method. Methods for detecting and measuring signals generated by detection labels are also known to those of skill in the art. For example, radioactive isotopes can be detected by scintillation counting or direct visualization; fluorescent molecules can be detected with fluorescent spectrophotometers; phosphorescent molecules can be detected with a spectrophotometer or directly visualized with a camera; enzymes can be detected by detection or visualization of the product of a reaction catalyzed by the enzyme; antibodies can be detected by detecting a secondary detection label coupled to the antibody. Such methods can be used directly in the disclosed method of amplification and detection. As used herein, detection molecules are molecules that interact with amplified nucleic acid and to which one or more detection labels are coupled.

L. Detection Probes

Detection probes are labeled oligonucleotides having sequence complementary to detection tags on TS-DNA or transcripts of TS-DNA. The complementary portion of a detection probe can be any length that supports specific and stable hybridization between the detection probe and the detection tag. For this purpose, a length of 10 to 35 nucleotides is preferred, with a complementary portion of a detection probe 16 to 20 nucleotides long being most preferred. Detection probes can contain any of the detection labels described elsewhere herein. Preferred labels are biotin and fluorescent molecules. A particularly preferred detection probe is a molecular beacon. Molecular beacons are detection probes labeled with fluorescent moieties where the fluorescent moieties fluoresce only when the detection probe is hybridized (Tyagi and Kramer, Nature Biotechnology 14:303-308 (1996)). The use of such probes eliminates the need for removal of unhybridized probes prior to label detection because the unhybridized detection probes will not produce a signal. This is especially useful in multiplex assays.

A preferred form of detection probe, referred to herein as a collapsing detection probe, contains two separate complementary portions. This allows each detection probe to hybridize to two detection tags in TS-DNA. In this way, the detection probe forms a bridge between different parts of the TS-DNA. The combined action of numerous collapsing detection probes hybridizing to TS-DNA will be to form a collapsed network of cross-linked TS-DNA. Collapsed TS-DNA occupies a much smaller volume than free, extended TS-DNA, and includes whatever detection label present on the detection probe. This result is a compact and discrete detectable signal for each TS-DNA. Collapsing TS-DNA is useful both for in situ hybridization applications and for multiplex detection because it allows detectable signals to be spatially separate even when closely packed. Collapsing TS-DNA is especially preferred for use with combinatorial multicolor coding.

TS-DNA collapse can also be accomplished through the use of ligand/ligand binding pairs (such as biotin and avidin) or hapten/antibody pairs. As described in U.S. Pat. No. 6,143,495 (Example 6), a nucleotide analog, BUDR, can be incorporated into TS-DNA during rolling circle replication. When biotinylated antibodies specific for BUDR and avidin are added, a cross-linked network of TS-DNA forms, bridged by avidin-biotin-antibody conjugates, and the TS-DNA collapses into a compact structure. Collapsing detection probes and biotin-mediated collapse can also be used together to collapse TS-DNA.

M. Address Probes

An address probe is an oligonucleotide having a sequence complementary to address tags on TS-DNA or transcripts of TS-DNA. The complementary portion of an address probe can be any length that supports specific and stable hybridization between the address probe and the address tag. For this purpose, a length of 10 to 35 nucleotides is preferred, with a complementary portion of an address probe 12 to 18 nucleotides long being most preferred. Address probe can contain a single complementary portion or multiple complementary portions. Preferably, address probes are coupled, either directly or via a spacer molecule, to a solid-state substrate or support.

N. Nucleic Acids

The disclosed methods and compositions use and involve nucleic acids, including, for example, base nucleic acids, manipulated product nucleic acids, mRNA, cDNA, primers, probes, amplification target circles, and other oligonucleotides. Nucleic acids are typically made up of nucleotides. These nucleic acids can be ribonucleic acids or deoxyribonucleic acids, or other types of nucleic acids. The nucleic acids can be modified in a number of ways, by for example having a capture tag attached to them, either through for example chemical coupling or enzymatic incorporation. The following is a brief discussion which is not meant to be limiting unless specified, but which illustrates the breadth of nucleic acids contemplated. It is also understood, however, that each specific embodiment of the nucleic acid based compositions including capture tags are individually and collectively contemplated. Thus, for example, an all DNA nucleic acid, an all RNA nucleic acid, an all DNA molecule except for a single biotin attached, and an all DNA molecule except for a single PNA linkage are contemplated as well as any other combination. It is also understood that each permutation or combination is individually herein disclosed and described even though each individual variation is not written down.

1. Nucleotide

A nucleotide is a molecule that contains a base moiety, a sugar moiety, and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is typically a ribose or a deoxyribose. The phosphate moiety of a nucleotide is typically pentavalent phosphate. Non-limiting examples of a nucleotides are 3′-AMP (3′-adenosine monophosphate) or 5′-GMP (5′-guanosine monophosphate).

2. Nucleotide Analog

A nucleotide analog is a nucleotide which contains some type of modification to either the base, sugar, or phosphate moieties. Modifications to the base moiety would include natural and synthetic modifications of A, C, G, and T/U as well as different purine or pyrimidine bases, such as uracil-5-yl (φ),hypoxanthin-9-yl (I), and 2-aminoadenin-9-yl. A modified base includes but is not limited to 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Additional base modifications can be found for example in U.S. Pat. No. 3,687,808, Englisch et al., Angewandte Chemie, International Edition, 1991, 30, 613, and Sanghvi, Y. S., Chapter 15, Antisense Research and Applications, pages 289-302, Crooke, S. T. and Lebleu, B. ed., CRC Press, 1993.

Often nucleotide analogs can have enhanced or additional properties to the nucleotide for which they are an analog. For example, 7-deaza-guanosine will form Watson-Crick interactions with cytidine, but because the N7 position of the guanine base is substituted with a C—H, interactions on the Hoogsteen face of the nucleotide analog are reduced. Often, polymers which have nucleotide analogs incorporated into them, are more stable with respect to degrading enzymes, such as exonucleases and RNase, than are polymers that incorporate the corresponding nucleotide. Certain nucleotide analogs, such as 5-substituted pyrimidines, 6-azapyrimidines and N-2, N-6 and O-6 substituted purines, including 2-aminopropyladenine, 5-propynyluracil and 5-propynylcytosine, and 5-methylcytosine can increase the stability of duplex formation. Base modifications can be combined with a sugar modification, such as 2′-O-methoxyethyl, for example, to achieve unique properties such as increased duplex stability. There are numerous United States patents such as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; and 5,681,941, which detail and describe a range of base modifications. Each of these patents is herein incorporated by reference.

Nucleotide analogs can also include modifications to the sugar moiety. Modifications to the sugar moiety would include natural modifications of the ribose and deoxy ribose as well as synthetic modifications. Sugar modifications include but are not limited to the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C1 to C10, alkyl or C2 to C10 alkenyl and alkynyl. 2′ sugar modifications also include but are not limited to —O[(CH₂)n O]m CH₃, —O(CH₂)n OCH₃, —O(CH₂)n NH₂, —O(CH₂)n CH₃, —O(CH₂)n —ONH₂ and —O(CH₂)nON[(CH₂)n CH₃)]₂, where n and m are from 1 to about 10.

Other modifications at the 2′ position include but are not limited to: C1 to C10 lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂, CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars would also include those that contain modifications at the bridging ring oxygen, such as CH2 and S. Nucleotide sugar analogs may also have sugar mimetics such as cyclobutyl moieties in place of the pentofuranosyl sugar. There are numerous United States patents that teach the preparation of such modified sugar structures such as U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. It is understood that these phosphate or modified phosphate linkages between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts and free acid forms are also included. Numerous United States patents teach how to make and use nucleotides containing modified phosphates and include but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

It is understood that nucleotide analogs need only contain a single modification, but may also contain multiple modifications within one of the moieties or between different moieties.

3. Nucleotide Substitutes

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid.

Nucleotide substitutes are nucleotides or nucleotide analogs that have had the phosphate moiety and/or sugar moieties replaced. Nucleotide substitutes do not contain a standard phosphorus atom. Substitutes for the phosphate can be for example, short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts. Numerous United States patents disclose how to make and use these types of phosphate replacements and include but are not limited to U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

It is also understood in a nucleotide substitute that both the sugar and the phosphate moieties of the nucleotide can be replaced, by for example an amide type linkage (aminoethylglycine) (PNA). U.S. Pat. Nos. 5,539,082; 5,714,331; and 5,719,262 teach how to make and use PNA molecules, each of which is herein incorporated by reference. (See also Nielsen et al., Science, 1991, 254, 1497-1500).

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937. Numerous United States patents teach the preparation of such conjugates and include, but are not limited to U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is understood that nucleic acids contain nucleotides or nucleotide analogs or nucleotide substitutes or nucleotide conjugates or any other type of nucleotide reagent in any combination collectively or individually and that all forms of nucleic acid manipulation capable of generating nucleic acids as contemplated herein are specifically contemplated. It is also understood that an oligonucleotide may typically contain any nucleotide, nucleotide analog, nucleotide substitute, nucleotide conjugate in any combination, and that wherever the word oligonucleotide is used all of the variations possible from nucleotide, nucleotide analog, nucleotide substitute, nucleotide conjugate are individually and collectively disclosed such that any combination is specifically herein disclosed.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH₂ or O) at the C6 position of purine nucleotides.

O. Oligonucleotide Synthesis

RT primers, rolling circle replication primers, detection probes, address probes, amplification target circles, DNA strand displacement primers, open circle probes, gap oligonucleotides, and any other oligonucleotides can be synthesized using established oligonucleotide synthesis methods. Methods to produce or synthesize oligonucleotides are well known. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

Many of the oligonucleotides described herein are designed to be complementary to certain portions of other oligonucleotides or nucleic acids such that stable hybrids can be formed between them. The stability of these hybrids can be calculated using known methods such as those described in Lesnick and Freier, Biochemistry 34:10807-10815 (1995), McGraw et al., Biotechniques 8:674-678 (1990), and Rychlik et al., Nucleic Acids Res. 18:6409-6412 (1990).

P. Open Circle Probes

Open circle probes (OCPs) are related to ATCs in that an open circle probe can become an ATC if specific enzymatic reactions are successfully completed. An open circle probe is a linear single-stranded DNA molecule, preferably containing between 50 to 1000 nucleotides, more preferably between about 60 to 150 nucleotides, and most preferably between about 70 to 100 nucleotides. The OCP has a 5′ phosphate group and a 3′ hydroxyl group. This allows the ends to be ligated (to each other or to other nucleic acid ends) using a ligase, coupled, or extended in a gap-filling operation. Preferred open circle probes for use in the disclosed method can form an intramolecular stem structure involving one or both of the OCP's ends. Such open circle probes are referred to herein as hairpin open circle probes. An intramolecular stem structure involving an end refers to a stem structure where the terminal nucleotides (that is, nucleotides at the end) of the OCP are hybridized to other nucleotides in the OCP.

Portions of the OCP have specific functions making the OCP useful for RCA and LM-RCA. These portions are referred to as the target probe portions, the primer complement portion, the spacer region, the detection tag portions, the secondary target sequence portions, the address tag portions, and the promoter portion. The target probe portions and the primer complement portion are required elements of an open circle probe. The primer complement portion is preferably part of the spacer region. Detection tag portions, secondary target sequence portions, and promoter portions are optional and, when present, are part of the spacer region. Address tag portions are optional and, when present, may be part of the spacer region. The primer complement portion, and the detection tag portions, the secondary target sequence portions, the address tag portions, and the promoter portion, if present, are preferably non-overlapping. However, various of these portions can be partially or completely overlapping if desired. Primer complement portions, spacer regions, detection tag portions, secondary target sequence portions, address tag portions, and promoter portions are generally the same, and have the same preferred features, configurations, and uses, as the same portions of amplification target circles as described elsewhere herein.

Generally, an open circle probe is a single-stranded, linear DNA molecule comprising, from 5′ end to 3′ end, a 5′ phosphate group, a right target probe portion, a spacer region, a left target probe portion, and a 3′ hydroxyl group, with a primer complement portion present as part of the spacer region. Those segments of the spacer region that do not correspond to a specific portion of the OCP can be arbitrarily chosen sequences. It is preferred that OCPs do not have any sequences that are self-complementary. It is considered that this condition is met if there are no complementary regions greater than six nucleotides long without a mismatch or gap. It is also preferred that OCPs containing a promoter portion do not have any sequences that resemble a transcription terminator, such as a run of eight or more thymidine nucleotides.

The open circle probe, when ligated and replicated, gives rise to a long DNA molecule containing multiple repeats of sequences complementary to the open circle probe. This long DNA molecule is referred to herein as tandem sequences DNA (TS-DNA). TS-DNA contains sequences complementary to the target probe portions, the primer complement portion, the spacer region, and, if present on the open circle probe, the detection tag portions, the secondary target sequence portions, the address tag portions, and the promoter portion. These sequences in the TS-DNA are referred to as target sequences (which match the original target sequence), primer sequences (which match the sequence of the rolling circle replication primer), spacer sequences (complementary to the spacer region), detection tags, secondary target sequences, address tags, and promoter sequences.

1. Target Probe Portions

There are two target probe portions on each OCP, one at each end of the OCP. The target probe portions can each be any length that supports specific and stable hybridization between the target probes and the target sequence. For this purpose, a length of 10 to 35 nucleotides for each target probe portion is preferred, with target probe portions 15 to 25 nucleotides long being most preferred. The target probe portion at the 3′ end of the OCP is referred to as the left target probe, and the target probe portion at the 5′ end of the OCP is referred to as the right target probe. These target probe portions are also referred to herein as left and right target probes or left and right probes. The target probe portions are complementary to a target nucleic acid sequence.

The target probe portions are complementary to the target sequence, such that upon hybridization the 5′ end of the right target probe portion and the 3′ end of the left target probe portion are base-paired to adjacent nucleotides in the target sequence, with the objective that they serve as a substrate for ligation.

Where the intramolecular stem structure of an open circle probe forms a stem and loop structure, it is preferred that a portion of one of the target probe portions of the open circle probe is in the loop of the stem and loop structure. This portion of the target probe portion in the loop can then hybridize to the target sequence of the open circle probe. Such an arrangement allows design of hairpin open circle probes where the stability of the intramolecular stem structure depends on the presence or absence of the specific target sequence. In particular, an open circle probe that forms a stem and loop structure with a portion of the target probe portion in the loop can be designed so that hybridization of the target probe portion in the loop to the target sequence disrupts the intramolecular stem structure (Tyagi and Kramer, Nat Biotechnol 14(3):303-8 (1996); Bonnet et al., Proc Natl Acad Sci USA 96(11):6171-6 (1999)). In this way, the intramolecular stem structure remains intact in the absence of the target sequence and thus reduces or eliminates the ability of the open circle probe to prime nucleic acid replication (or to serve as a template for rolling circle replication). Preferably, the hybrid between the target sequence and the target probe portion at the end of the open circle probe is more stable than the intramolecular stem structure. This helps stabilize hybridization of the open circle probe to the target sequence in competition with the intramolecular stem structure.

In another form of open circle probe, the 5′ end and the 3′ end of the target probe portions may hybridize in such a way that they are separated by a gap space. In this case the 5′ end and the 3′ end of the OCP may only be ligated if one or more additional oligonucleotides, referred to as gap oligonucleotides, are used, or if the gap space is filled during the ligation operation. The gap oligonucleotides hybridize to the target sequence in the gap space to a form continuous probe/target hybrid. The gap space may be any length desired but is generally ten nucleotides or less. It is preferred that the gap space is between about three to ten nucleotides in length, with a gap space of four to eight nucleotides in length being most preferred. Alternatively, a gap space could be filled using a DNA polymerase during the ligation operation. When using such a gap-filling operation, a gap space of three to five nucleotides in length is most preferred. As another alternative, the gap space can be partially bridged by one or more gap oligonucleotides, with the remainder of the gap filled using DNA polymerase.

Q. Gap Oligonucleotides

Gap oligonucleotides are oligonucleotides that are complementary to all or a part of that portion of a nucleotide sequence, such as a target sequence, which covers a gap space between the ends of hybridized probes (the ends of open circle probes, for example). Gap oligonucleotides have a phosphate group at their 5′ ends and a hydroxyl group at their 3′ ends. This facilitates ligation of gap oligonucleotides to probes, or to other gap oligonucleotides. The gap space between the ends of hybridized probes can be filled with a single gap oligonucleotide, or it can be filled with multiple gap oligonucleotides. For example, two 3 nucleotide gap oligonucleotides can be used to fill a six nucleotide gap space, or a three nucleotide gap oligonucleotide and a four nucleotide gap oligonucleotide can be used to fill a seven nucleotide gap space. Gap oligonucleotides are particularly useful for distinguishing between closely related target sequences. For example, multiple gap oligonucleotides can be used to amplify different allelic variants of a target sequence. By placing the region of the target sequence in which the variation occurs in the gap space formed by an open circle probe, a single open circle probe can be used to amplify each of the individual variants by using an appropriate set of gap oligonucleotides.

R. DNA Polymerases

DNA polymerases useful in rolling circle replication must perform rolling circle replication of primed single-stranded circles. Such polymerases are referred to herein as rolling circle DNA polymerases. For rolling circle replication, it is preferred that a DNA polymerase be capable of displacing the strand complementary to the template strand, termed strand displacement, and lack a 5′ to 3′ exonuclease activity. Strand displacement is necessary to result in synthesis of multiple tandem copies of the ligated OCP. A 5′ to 3′ exonuclease activity, if present, might result in the destruction of the synthesized strand. DNA polymerases for use in the disclosed method can also be highly processive, if desired. The suitability of a DNA polymerase for use in the disclosed method can be readily determined by assessing its ability to carry out rolling circle replication. Preferred rolling circle DNA polymerases are Bst DNA polymerase, VENT® DNA polymerase (Kong et al., J. Biol. Chem. 268:1965-1975 (1993)), ThermoSequenase™, delta Tts DNA polymerase, bacteriophage φ29 DNA polymerase (U.S. Pat. Nos. 5,198,543 and 5,001,050 to Blanco et al.), phage M2 DNA polymerase (Matsumoto et al., Gene 84:247 (1989)), phage φPRD1 DNA polymerase (Jung et al., Proc. Natl. Acad. Sci. USA 84:8287 (1987)), Klenow fragment of DNA polymerase I (Jacobsen et al., Eur. J. Biochem. 45:623-627 (1974)), T5 DNA polymerase (Chatterjee et al., Gene 97:13-19 (1991)), PRD1 DNA polymerase (Zhu and Ito, Biochim. Biophys. Acta. 1219:267-276 (1994)), modified T7 DNA polymerase (Tabor and Richardson, J. Biol. Chem. 262:15330-15333 (1987); Tabor and Richardson, J. Biol. Chem. 264:6447-6458 (1989); Sequenase™ (U.S. Biochemicals)), and T4 DNA polymerase holoenzyme (Kaboord and Benkovic, Curr. Biol. 5:149-157 (1995)). More preferred are Bst DNA polymerase, VENT® DNA polymerase, ThermoSequenase™, and delta Tts DNA polymerase. Bst DNA polymerase is most preferred.

Strand displacement can be facilitated through the use of a strand displacement factor, such as helicase. It is considered that any DNA polymerase that can perform rolling circle replication in the presence of a strand displacement factor is suitable for use in the disclosed method, even if the DNA polymerase does not perform rolling circle replication in the absence of such a factor. Strand displacement factors useful in the disclosed method include BMRF1 polymerase accessory subunit (Tsurumi et al., J. Virology 67(12):7648-7653 (1993)), adenovirus DNA-binding protein (Zijderveld and van der Vliet, J. Virology 68(2): 1158-1164 (1994)), herpes simplex viral protein ICP8 (Boehmer and Lehman, J. Virology 67(2):711-715 (1993); Skaliter and Lehman, Proc. Natl. Acad. Sci. USA 91(22):10665-10669 (1994)), single-stranded DNA binding proteins (SSB; Rigler and Romano, J. Biol. Chem. 270:8910-8919 (1995)), and calf thymus helicase (Siegel et al., J. Biol. Chem. 267:13629-13635 (1992)).

The ability of a polymerase to carry out rolling circle replication can be determined by using the polymerase in a rolling circle replication assay such as those described in Fire and Xu, Proc. Natl. Acad. Sci. USA 92:4641-4645 (1995) and in U.S. Pat. No. 6,143,495 (Example 1).

Another type of DNA polymerase can be used if a gap-filling synthesis step is used. When using a DNA polymerase to fill gaps, strand displacement by the DNA polymerase is undesirable. Such DNA polymerases are referred to herein as gap-filling DNA polymerases. Unless otherwise indicated, a DNA polymerase referred to herein without specifying it as a rolling circle DNA polymerase or a gap-filling DNA polymerase, is understood to be a rolling circle DNA polymerase and not a gap-filling DNA polymerase. Preferred gap-filling DNA polymerases are T7 DNA polymerase (Studier et al., Methods Enzymol. 185:60-89 (1990)), DEEP VENT® DNA polymerase (New England Biolabs, Beverly, Mass.), modified T7 DNA polymerase (Tabor and Richardson, J. Biol. Chem. 262:15330-15333 (1987); Tabor and Richardson, J. Biol. Chem. 264:6447-6458 (1989); Sequenase™ (U.S. Biochemicals)), and T4 DNA polymerase (Kunkel et al., Methods Enzymol. 154:367-382 (1987)). An especially preferred type of gap-filling DNA polymerase is the Thermus flavus DNA polymerase (MBR, Milwaukee, Wis.). The most preferred gap-filling DNA polymerase is the Stoffel fragment of Taq DNA polymerase (Lawyer et al., PCR Methods Appl. 2(4):275-287 (1993), King et al., J. Biol. Chem. 269(18):13061-13064 (1994)).

The ability of a polymerase to fill gaps can be determined by performing gap-filling LM-RCA. Gap-filling LM-RCA is performed with an open circle probe that forms a gap space when hybridized to the target sequence. Ligation can only occur when the gap space is filled by the DNA polymerase. If gap-filling occurs, TS-DNA can be detected, otherwise it can be concluded that the DNA polymerase, or the reaction conditions, is not useful as a gap-filling DNA polymerase.

S. RNA Polymerases

Any RNA polymerase which can carry out transcription in vitro and for which promoter sequences have been identified can be used in the disclosed rolling circle transcription method. Stable RNA polymerases without complex requirements are preferred. Most preferred are T7 RNA polymerase (Davanloo et al., Proc. Natl. Acad. Sci. USA 81:2035-2039 (1984)) and SP6 RNA polymerase (Butler and Chamberlin, J. Biol. Chem. 257:5772-5778 (1982)) which are highly specific for particular promoter sequences (Schenborn and Meirendorf, Nucleic Acids Research 13:6223-6236 (1985)). Other RNA polymerases with this characteristic are also preferred. Because promoter sequences are generally recognized by specific RNA polymerases, the OCP or ATC should contain a promoter sequence recognized by the RNA polymerase that is used. Numerous promoter sequences are known and any suitable RNA polymerase having an identified promoter sequence can be used. Promoter sequences for RNA polymerases can be identified using established techniques.

T. DNA Ligases

Any DNA ligase is suitable for use in the disclosed methods. Preferred ligases are those that preferentially form phosphodiester bonds at nicks in double-stranded DNA. That is, ligases that fail to ligate the free ends of single-stranded DNA at a significant rate are preferred. Thermostable ligases are especially preferred. Many suitable ligases are known, such as T4 DNA ligase (Davis et al., Advanced Bacterial Genetics—A Manual for Genetic Engineering (Cold Spring Harbor Laboratory, Cold Spring Harbor, N.Y., 1980)), E. coli DNA ligase (Panasnko et al., J. Biol. Chem. 253:4590-4592 (1978)), AMPLIGASE® (Kalin et al., Mutat. Res., 283(2):119-123 (1992); Winn-Deen et al., Mol Cell Probes (England) 7(3):179-186 (1993)), Taq DNA ligase (Barany, Proc. Natl. Acad. Sci. USA 88:189-193 (1991), Thermus thermophilus DNA ligase (Abbott Laboratories), Thermus scotoductus DNA ligase and Rhodothermus marinus DNA ligase (Thorbjarnardottir et al., Gene 151:177-180 (1995)). T4 DNA ligase is preferred for ligations involving probes hybridized to RNA sequences due to its ability to ligate DNA ends involved in DNA:RNA hybrids (Hsuih et al., Quantitative detection of HCV RNA using novel ligation-dependent polymerase chain reaction, American Association for the Study of Liver Diseases (Chicago, Ill, Nov. 3-7, 1995)).

The frequency of non-target-directed ligation catalyzed by a ligase can be determined as follows. LM-RCA is performed with an open circle probe and a gap oligonucleotide in the presence of a target sequence. Non-targeted-directed ligation products can then be detected by using an address probe specific for the open circle probe ligated without the gap oligonucleotide to capture TS-DNA from such ligated probes. Target-directed ligation products can be detected by using an address probe specific for the open circle probe ligated with the gap oligonucleotide. By using a solid-state substrate with regions containing each of these address probes, both target-directed and non-target-directed ligation products can be detected and quantitated. The ratio of target-directed and non-target-directed TS-DNA produced provides a measure of the specificity of the ligation operation. Target-directed ligation can also be assessed as discussed in Barany (1991).

U. Substrates

Substrates can be used in the disclosed method as a solid support for components used in the method, preferably capture probes. For example, one or more of the components of the method can be adhered to or coupled to a substrate. This can allow simplified washing and handling of the components, can allow automation of all or part of the method, and allows identification of molecules by virtue of their association with particular locations on the substrate. It is preferred that capture probes be captured, adhered to, or otherwise coupled to a substrate. “Substrate” and “support” are used interchangeably herein to refer to solid-state compositions.

Substrates for use in the disclosed method can include any solid material to which components of the assay can be adhered or coupled. This includes materials such as acrylamide, cellulose, nitrocellulose, glass, polystyrene, polyethylene vinyl acetate, polypropylene, polymethacrylate, polyethylene, polyethylene oxide, glass, polysilicates, polycarbonates, teflon, fluorocarbons, nylon, silicon rubber, polyanhydrides, polyglycolic acid, polylactic acid, polyorthoesters, polypropylfumerate, collagen, glycosaminoglycans, and polyamino acids. Substrates can have any useful form including thin films or membranes, beads, bottles, dishes, fibers, optical fibers, woven fibers, shaped polymers, particles and microparticles. Preferred forms of substrates are plates and beads. The most preferred form of beads are magnetic beads.

Methods for immobilization of oligonucleotides, such as capture probes, to substrates are well established. Oligonucleotides, including oligonucleotide capture probes, can be coupled to substrates using established coupling methods. For example, suitable attachment methods are described by Pease et al., Proc. Natl. Acad. Sci. USA 91(11):5022-5026 (1994), and Khrapko et al., Mol Biol (Mosk) (USSR) 25:718-730 (1991). A method for immobilization of 3′-amine oligonucleotides on casein-coated slides is described by Stimpson et al., Proc. Natl. Acad. Sci. USA 92:6379-6383 (1995). A preferred method of attaching oligonucleotides to solid-state substrates is described by Guo et al., Nucleic Acids Res. 22:5456-5465 (1994).

Adhering or coupling components to a substrate is preferably accomplished by adhering or coupling capture tags to the substrate. The capture tags can then mediate adherence of a component, such as a capture probe, by binding to, or interacting with, a capture tag on the component. Capture tags immobilized on a substrate allow capture of the molecules. Such capture provides a convenient means of washing away reaction components that might interfere with subsequent detection steps. By attaching different capture tags to different regions of a solid-state substrate, different components can be captured at different, and therefore diagnostic, locations on the substrate. For example, in a microtiter plate multiplex assay, capture tags specific for up to 96 different components can be immobilized on a microtiter plate, each in a different well. Capture and detection will occur only in those wells corresponding to the capture tag for which the corresponding component, such as RNA molecules, were present in a sample.

V. Kits

The materials described above can be packaged together in any suitable combination as a kit useful for performing the disclosed method. It is preferred that the kit components in a given kit be designed and adapted for use together in the disclosed method. For example disclosed are kits for amplifying messenger RNA, the kit comprising one or more amplification target circles and one or more RT primers. The amplification target circles preferably each comprise a single-stranded, circular DNA molecule comprising a primer complement portion. In one form, the RT primers can each comprise a reverse transcription primer portion and a rolling circle replication primer portion, wherein the reverse transcription primer portion and the rolling circle replication primer portion each comprise a 5′ end, wherein the reverse transcription primer portion and the rolling circle replication primer portion are not linked via their 5′ ends, wherein both the reverse transcription primer portion and the rolling circle replication primer portion can prime nucleic acid replication, wherein the rolling circle replication primer portion is complementary to a portion of one or more amplification target circles. The reverse transcription primer portion of the RT primers can comprise poly T. Preferred kits also contain one or more capture probes, wherein each capture probe comprises a sequence matching all or a portion of the sequence of messenger RNA molecules of interest.

The disclosed kits can also include one or more secondary DNA strand displacement primers, one or more tertiary DNA strand displacement primer, one or more open circle probes, one or more gap oligonucleotides, and/or one or more detection probes. Preferably, a portion of each of the detection probes in a kit has sequence matching or complementary to a portion of a different one of the amplification target circles in that kit.

A preferred kit for selectively manipulating and detecting one or more nucleic acid molecules can include one or more RT primers, one or more amplification target circles, one or more rolling circle replication primers, and one or more capture probes. In this kit, it is preferred that the capture probes are immobilized on a solid substrate or support. It is also preferred that the RT primers, the rolling circle replication primers, or both comprise capture tags.

W. Mixtures

Disclosed are mixtures formed by performing any of the disclosed methods. For example, disclosed are mixtures comprising cDNA strands, a set of capture probes, one or more rolling circle replication primers, and one or more amplification target circles. Preferred mixtures comprise (a) cDNA strands produced by incubating one or more RT primers with a nucleic acid sample and reverse transcribing, wherein each cDNA strand comprises one of the RT primers, wherein each RT primer comprises a reverse transcription primer portion, (b) a set of capture probes hybridized to the cDNA strands, (c) one or more rolling circle replication primers associated with the cDNA strands, wherein the rolling circle replication primers each comprise a capture tag, and wherein the association occurs via the capture tag, (d) one or more amplification target circles associated with the rolling circle replication primers.

Whenever the method involves mixing compositions or components or reagents for example, performing the method creates a number of different mixtures. For example, if the method includes 3 mixing steps, after each one of these steps a unique mixture is formed if the steps are performed sequentially. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### Uses

The disclosed methods and compositions are applicable to numerous areas including, but not limited to, analysis of nucleic acids present in a sample (for example, analysis of messenger RNA in a sample), disease detection, mutation detection, gene expression profiling, RNA expression profiling, gene discovery, gene mapping (molecular haplotyping), agricultural research, and virus detection. The preferred use of the disclosed method is analysis of messenger RNA expression. Other uses include detection of nucleic acids in situ in cells, on microarrays, on DNA fibers, and on genomic DNA arrays; detection of RNA in cells; RNA expression profiling; molecular haplotyping; mutation detection; detection of abnormal RNA (for example, overexpression of an oncogene or absence of expression of a tumor suppressor gene); expression in cancer cells; detection of viral genome in cells; viral RNA expression; detection of inherited diseases such as cystic fibrosis, muscular dystrophy, diabetes, hemophilia, sickle cell anemia; assessment of predisposition for cancers such as prostate cancer, breast cancer, lung cancer, colon cancer, ovarian cancer, testicular cancer, pancreatic cancer.

### EXAMPLES

The following examples are set forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (for example, amounts, temperature, etc.), but some errors and deviations should be accounted for. Unless indicated otherwise, parts are parts by weight, temperature is in ° C. or is at ambient temperature, and pressure is at or near atmospheric.

### A. Example 1

Cell Culture and Preparation of PolyA+ mRNA from Cells

The fibroblast cell line, CRL2091, was purchased from the ATCC (Manassas, Va.). Cells were grown in Dulbecco's Modified Eagle's Medium (DMEM) with 10 mg/ml glucose, containing 10% (vol/vol) fetal bovine serum (FBS; Life Technologies, Carlsbad, Calif.)×10 U/ml Penicillin and 10 μg/ml Streptomycin. Cells were allowed to grow to 70% confluence and harvested by trypsinization. Three milliliters FBS was added to neutralize the trypsin and the cells were collected by centrifugation in a tabletop centrifuge at 1,000×g. The cell pellet was washed by resuspending in phosphate buffered saline (PBS) and used for preparation of polyA+ mRNA. Approximately 1 million cells per T-175 flask was routinely obtained. Approximately 80-100 million cells were used for preparation of polyA+ mRNA. Jurkat cells were grown in a same manner, except that RPMI medium was used instead of DMEM. Prostate and Placental mRNA were purchased from Clontech (Palo Alto, Calif.).

PolyA+ mRNA was extracted from the cell pellet using the FastTrack 2.0 mRNA isolation kit, as described by the manufacturer (Invitrogen; Valencia, Calif.). Cells were resuspended in 20 ml extraction buffer and vortexed for 20 seconds prior to performing the isolation.

### B. Example 2

cDNA Preparation and Purification

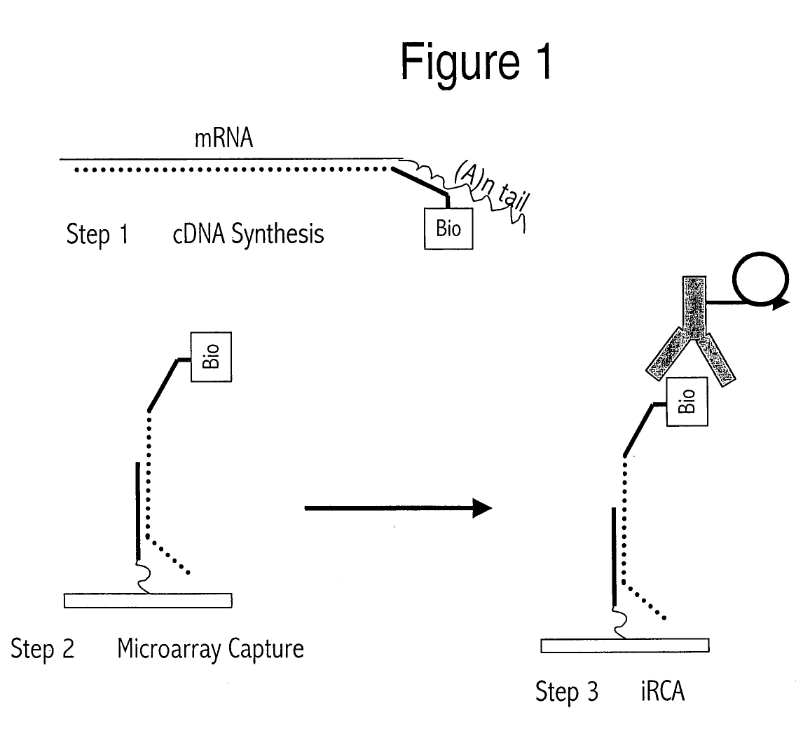

This example involves synthesis of cDNA of the form illustrated in FIG. 1 (with a biotin capture tag as part of the RT primer that is incorporated into the cDNA strand). cDNA was prepared from 0.5 μg of mRNA according to methods know in the art (Molecular Cloning, Maniatis et al, CSHL Press, Cold Spring Harbor, N.Y.).

1. Synthesis of Biotin-labeled cDNA

The following components were assembled in a 0.2 ml PCR tube.

| | |
|---|---|
| Reagents | Volume |

| | | |
|---|---|---|
| 5X First Strand reaction buffer | 6 | μl |
| 0.1 M DTT | 3 | μl |
| dNTP mix | 0.6 | μl |
| (10 mM dTTP, 25 mM each of | | |
| dCTP, dGTP, dATP) | | |
| 1 mM Cy5 dUTP | 3 | μl |
| 5′-Biotin Oligo (dT)₂₀ | 2 | μl |
| (MSI-1060, 1 μg/μl) | | |
| mRNA | 0.5 | μg |
| Autoclaved water | to 29 | μl |

2. Synthesis of cDNA Containing a Biotin Capture Tag at the 5′-end

The following reaction mix was assembled in a 0.2 ml PCR tube:

| | |
|---|---|
| Reagents | Volume |

| | | |
|---|---|---|
| 5X First strand buffer | 6 | μl |
| 0.1 M DTT | 3 | μl |
| dNTP mix | 0.6 | μl |
| (25 mM each of | | |
| dCTP, dGTP, TTP, dATP) | | |
| 5′-Biotin Oligo (dT)₂₀ | 2 | μl |
| (MSI-1060, 1 μg/μl) | | |
| mRNA | 0.5 | μg |
| Autoclaved water | to 29 | μl |

The biotin oligo (dT)₂₀ is the RT primer with a biotin capture tag.

3. Annealing Oligo(dT) Primer to mRNA.

The reaction was mixed by vortexing. The reaction tube was placed in a thermal cycler. Program “cDNA” in 96 well MJ Research “DNA Engine” thermal cycler was run to anneal primer to mRNA. The cycles were: 85° C. 2 min, 80° C. 2 min, 78° C. 2 min, 75° C. 2 min, 70° C. 2 min, 65° C. 2 min, 60° C. 2 min, 58° C. 2 min, 55° C. 2 min, 50° C. 2 min, 48° C. 2 min, 45° C. 2 min, 43° C. 2 min, 42° C. Indefinitely

4. First Strand cDNA Synthesis.

When the temperature of the thermal cycler reached 42° C., 1 μl Superscript II reverse transcriptase (200 U/μl; Invitrogen, Carlsbad, Calif.) was added. The reaction was then incubated at 42° C. for 1 hour. Another aliquot of 1 μl Superscript II (200 U/μl stock) was then added and the reaction was incubated at 42° C. for 1 hour.

5. Purification of cDNA

To the tube containing cDNA, 15 μl of 0.1N NaOH was added. The reaction was then incubated at 65° C. for 10 min. The reaction was neutralized by added 10 μl 0.01N HCl and 10 μl 2 M Sodium Acetate, pH 5.2 to the reaction and mixing by vortexing. The reaction was applied to a QLAquick spin column (QIAgen). The purification protocol was followed as per QIAGEN Manual. The DNA sample was then from the column with 60 μl water.

### C. Example 3

Microarray Preparation

This example involves production of a solid substrate with capture probes attached. Plain glass slides were derivatized as described (Guo et al, Nucleic Acids Res. 22:5456-5465 (1994)). Gold Seal Amino-Silane slides (Fisher Scientific) were placed in a glass slide staining rack and washed for 10 minutes in an Ultrasonic cleaner containing a 1:10 dilution of the Ultrasonic Cleaning solution. Slides were next soaked by shaking on an orbital shaker for 1 hour at room temperature in 25% Ammonium Hydroxide and rinsed with Milli-Q water and in 100% ethanol for 2 minutes each at room temperature.

Slides were incubated in 2% aminopropyltriethoxysilane solution in ethanol for 2 hours at room temperature with shaking on an orbital shaker and rinsed in ethanol for 2 minutes. The slides were cured overnight by placing in a vacuum oven at 110° C. in a vacuumed environment (20 psi).

The amino-silane-coated slides were next treated with 1,4-phenylene diisothiocyanate (PDITC). All steps were perform in a chemical hood. A solution of 0.2% PDITC (1,4-phenylene diisothiocyanate) in a 10% solution of pyridine in DMF (dimethyl formamide) was prepared in a large beaker. The PDITC solution was poured into jars containing slides and the jars were placed on an orbital shaker (speed 2-3) for 2 hours at room temperature. The slides were then washed twice with dichloroethane for 3 minutes. After final wash, place all slide racks onto a paper towel. Nitrogen tank was turned on to obtain a flow of 10-15 CFH. Slides were dried with stream of nitrogen. Slides were placed in slide boxes and the boxes were placed in a dessicator. The dessicator was put into 4° C. refrigerator for storage. Slides may be stored indefinitely before microarraying.

PDITC coated slides were microarrayed using a GeneMachines Omnigrid Printing Robot and fitted with micromachined print heads from Majer Scientific. The slides were placed on the arrayer platform. Oligonucleotides (capture probes) derivatized with a 3′ or 5′ amino group were dissolved at 0.1 μM in 100 mM Sodium phosphate, pH9.0, aliquoted into a 96-well plate and placed on the arrayer platform. Microarraying of the oligonucleotides on the slides was performed as per the arrayer manufacturer's recommendations. Oligonucleotides were either synthesized in-house or purchased from Integrated DNA Technologies, Coralville, Iowa and were purified by HPLC.

The structures of the capture probes were:

| SEQ ID NO. | ID | GenBank ID | Capture Probe Structure |
|---|---|---|---|
| SEQ ID NO:1 | RG4.3A | P55-C-FOS PROTO-ONCOGENE PROTEIN | 5′-NH2-C12-AAA AAA AAA AAA AAA |
| | | (R12840) | CCAGAAGAGATGTCTGTGG |
| SEQ ID NO:2 | RG4.3B | MAP KINASE PHOSPHATASE-1 | 5′-NH2-C12-AAA AAA AAA AAA AAA |
| | | (W90037) | GGTGATGACTTAGCGTCA AG |
| SEQ ID NO:3 | RG4.3C | Early growth response protein 1 | 5′-NH2-C12-AAA AAA AAA AAA AAA |
| | | (H27638) | GTTTAAAAAGTTTCACGTCTTG |
| SEQ ID NO:4 | RG4.6A | Interleukin 6 | 5′-NH2-C12-AAA AAA AAA AAA AAA |
| | | (B cell stimulatory factor 2) | CTGCAGGACATGACAACTC |
| | | (W31016) | |
| SEQ ID NO:5 | RG4.6B | Myeloid cell leukemia sequence 1 | 5′-NH2-C12-AAA AAA AAA AAA AAA |
| | | (BCL2-related) | GTAATTAGGAACCTGTTTCTTAC |
| | | (R77346) | |
| SEQ ID NO:6 | RG4.6C | Jun B proto-oncogene | 5′-NH2-C12-AAA AAA AAA AAA AAA |
| | | (W30678) | CTTCTGAACGTCCCCTGC |
| SEQ ID NO:7 | RG4.12A | Interleukin 8 (W40283) | 5′-NH2-C12-AAA AAA AAA AAA AAA |
| | | | GAAGATGAATCATTGATTGAATA |
| SEQ ID NO:8 | RG4.12B | Activating transcription factor 3 | 5′-NH2-C12-AAA AAA AAA AAA AAA |
| | | (ATF3) (AA004917) | CGTTAACACAAAATCCATGGG |
| SEQ ID NO:9 | RG4.12C | Inhibitor of DNA binding 3, (ID3) | 5′-NH2-C12-AAA AAA AAA AAA AAA |
| | | (W46413) | ACGACAAAAGGAGCTTTTGC |
| SEQ ID NO:10 | RG0.33A | SID297445 Homo sapiens DNA | 5′-NH2-C12-AAA AAA AAA AAA AAA |
| | | recombination and (W03632) | GGTCTCAAAGAGGAAGAGC |
| SEQ ID NO:11 | RG0.33B | H. sapiens DAP-kinase mRNA | 5′-NH2-C12-AAA AAA AAA AAA AAA |
| | | (AA024655) | GCTCTAGGAAGACATTTTTCC |
| SEQ ID NO:12 | RG0.33C | SERUM AMYLOID A PROTEIN PRECURSOR | 5′-NH2-C12-AAA AAA AAA AAA AAA |
| | | (H25590) | CCAGAGAGAATATCCAGAGAT |
| SEQ ID NO:13 | RG0.36A | CD38 antigen (p45) | 5′-NH2-C12-AAA AAA AAA AAA AAA |
| | | (R98115) | CCATGTGATGCTCAATGGAT |
| SEQ ID NO:14 | RG0.312A | SID51402 Homo sapiens | 5′-NH2-C12-AAA AAA AAA AAA AAA |
| | | monocyte/macrophage (H19389) | GATTTCCAACATCCTGCAGG |
| SEQ ID NO:15 | RG1.0A | *Superoxide dismutase 2 | 5′-NH2-C12-AAA AAA AAA AAA AAA |
| | | mitochondrial | CAAGTTTAAGGAGAAGCTGAC |
| SEQ ID NO:16 | RG1.0B | Cyclin B1 | 5′-NH2-C12-AAA AAA AAA AAA AAA |
| | | | GATTCTAAGAGCTTTAAACTTTG |
| SEQ ID NO:17 | RG1.0C | SID49950 FLAP ENDONUCLEASE-1 | 5′-NH2-C12-AAA AAA AAA AAA AAA |
| | | | CAGTTTAATGGACACTAAGTC |
| SEQ ID NO:18 | RG1.0D | Homo sapiens serine/threonine | 5′-NH2-C12-AAA AAA AAA AAA AAA |
| | | kinase (BTAK) mRNA, complete cds | CTACTTATACTGGTTCATAATC |
| SEQ ID NO:19 | RG-M-PM | M1101K-PM | 5′-NH2-C12-TTTGGAACCAGCGCAGTGT |
| | | | TGACAGGTACAAGAACCAGTT |
| SEQ ID NO:20 | RG-M-MM | M1101K-MM | 5′-NH2-C12-TTTGGAACCAGCGCAGTGT |
| | | | TGACAGGTACAAGAACCAGTA |
| SEQ ID NO:21 | RG-G-PM | G542X-PM | 5′-NH2-C12-GAA CTA TAT TGT CTT |
| | | | TCT CTG CAA ACT TGG AGA TGT CC |
| SEQ ID NO:22 | RG-G-MM | G542X-MM | 5′-NH2-C12-GAA CTA TAT TGT CTT |
| | | | TCT CTG CAA ACT TGG AGA TGT CG |

Slides were cured for 4-12 hours on the arrayer platform following deposition of the oligonucleotides. Microarryed slides were deactivated by incubating in 0.5 mM glycine solution for 30 minutes at 37° C. and blocked for 1 hr in a solution containing 50 mM glycine, (pH 9.5) and 3% bovine serum albumin (BSA). The slides were then washed once in PBS/0.1% Tween-20, and rinsed briefly in water.

Prior to hybridization, slides were prehybridized in the following solution:

| Reagents | Volume (μl) |
|---|---|
| 20X SSC | 8 |
| Yeast tRNA (10 μg/μl) | 1 |
| Sonicated Herring Sperm DNA | 1 |
| (10 μg/μl stock stored frozen) | |
| Human Cot I DNA (1 μg/μl) | 10 |
| 10% Tween-20 | 0.4 |
| Water | 59.6 |

80 μl of prehybridization mix was applied to each subarray on the slide in a Titanium Hybridization Chamber and incubated for one hour at 50° C. in a Flattop PCR instrument (MJ Research). Slides were washed inside the titanium chamber once with 3×SSCT and once with 1×SSC.

### D. Example 4

Hybridization of cDNA to Capture Probes on Microarrays

Each subarray was hybridized in 80 μl of a solution containing

| Reagents | Volume (μl) |
|---|---|
| 20X SSC | 8 |
| Yeast tRNA (10 μg/μl) | 1 |
| Sonicated Herring Sperm DNA | 1 |
| (10 μg/μl) | |
| Human Cot I DNA (1 μg/μl) | 5 |
| 10% Tween-20 | 0.4 |
| MSI-403 | (250 nM final) |
| MSI-405 | (250 nM final) |
| Water | to 20 μl |

The sequence of MSI-403 was 5′-Biotin-GGACATCTCCAAGTTTGCAGA GAAAGACAATATAGTTCTT-Biotin-3′ (SEQ ID NO:23) and of MSI-405 was 5′-Biotin-AACTGGTTCTTGTACCTGTCAACACTGCG CTGGTTCCAAA-Biotin -3′ (SEQ ID NO:24).

The reaction was mixed by vortexing. The targets were allowed to hybridize with the probes in the microarray at 50° C. for 18 hours in the titanium chamber.

Slides were washed with 3×SSCT inside the titanium chamber. The chamber was disassembled and the slides placed in a 50 ml screw cap tube. The slides were washed with 2×SSC, 0.05% Tween-20 for 10 minutes at room temperature with gentle agitation followed by 1×SSC for 10 minute. The slides were then washed with 0.05% SSC at room temperature with agitation for 1 min. The buffer was discarded and the wash repeated. The slides were placed on clean, dry Kimwipes in plate centrifuge. The slides were then spun at 1,000 rpm for 2 minutes.

### E. Example 5

RCA Signal Amplification

This example involves association of rolling circle replication primer with cDNA strands where the association occurs via biotin capture tags in the cDNA strands and Neutravidin capture tags in the rolling circle replication primers. The resulting tandem sequence DNA was detected using detection probes labeled with Cy5. The amplification target circle (Circle 1) and conjugate comprising the rolling circle replication primer (Primer 1 (Pr1)) was pre-annealed by mixing 800 ng of Neutravidin-Pr1 conjugate and 50 nM Circle 1 in 80 μl of 1×PBS, 0.05% Tween-20. The sequence of Circle 1 is: 5′-CTC AGC TGT GTA ACA ACA TGA AGA TTG TAG GTC AGA ACT CAC CTG TTA GAA ACT GTG AAG ATC GCT TAT TAT GTC CTA TC-3′ (SEQ ID NO:25) and the sequence of Primer 1 is: 5′-NH₂-(Carbon)₁₂-(A)₅₀-ACACAGCTGAGGATAGGACATAATAAGC-3′ (SEQ ID NO:26).

The reaction was incubated at 37° C. for 30 min. The area around each subarray was marked using a Pap pen and allowed to dry. 80 μl pre-annealed conjugate was applied to each subarray. The reaction was then incubated at 37° C. for 30 min. The slide was washed three times with 1×PBS, 0.05% Tween-20, 2 min each at room temperature with agitation.

RCA mix was prepared as follows:

| Reagents | Volume (μl) |
|---|---|
| Autoclaved water | 63.8 |
| 10X φ29 Reaction Buffer | 8 |
| (10X stock = 500 mM Tris-HCl, pH 7.9, | |
| 100 mM MgCl2, 100 mM Ammonium | |
| Sulfate, 2 mg/ml BSA) | |
| 10 mM each dNTPs | 8 |
| φ29 DNA Polymerase (80 U/μl) | 0.2 |

80 μl RCA mix was added to each subarray. The slides were incubated for 1 hour at 30° C. by placing them in a petri dish containing moist Kimwipes. Slides were washed three times with 2×SSC, 0.05% Tween-20, 2 min at room temperature with agitation.

The preferred sequence of a detection probe based on the sequence of Primer 1 and labeled with a Cy5 fluorophore tag is: 5′-Cy5-TGT CCT ATC CTC AGC TGG-Cy5-3′ (SEQ ID NO:27). 80 μl detection probe mix (0.5 μM detection probe in 2×SSC, 0.05% Tween-20) was added to each subarray and incubated for 30 min at 37° C. in petri dish containing moist Kimwipes. Slides were washed with 3 changes of 2×SSC, 0.05% Tween-20 for 2 min at room temperature with agitation. Slides were spun dry. Slides were scanned in Axon 4000B (ScanArray4000LITE or equivalent scanner can be used) at 635 nM. The preferred PMT setting is 600.

### F. Example 6

Signal Amplification of mRNA from Human Placenta

Human Placental cDNA labeled with Cy5 or unlabeled cDNA primed with an oligo(dT) containing a biotin tag at the 5′ end were prepared as described in Examples 1 through 3, and hybridized to microarrays as described in Example 4. After washing away unhybridized cDNA, the slides were either scanned directly (Cy5 cDNA) or after performing immunoRCA as described in Example 5. The result was increased signal intensity by RCA signal amplification compared to that with Cy5-labeled cDNA. There were 25 to 50 Cy5-UTP labels per cDNA fragment on the RCA slides compared with a single biotin tag per cDNA (and thus a single Cy5 label) in the Cy5-labeled cDNA. The non-specific background was negligible.

### G. Example 7

Signal Amplification of mRNA from Serum-treated Human Fibroblast Cells

RNA from human Fibroblast cells treated with fetal bovine serum for 30 min post starvation was processed as in Example 6. The result was increased signal intensity with RCA signal amplification compared to that with Cy5-labeled cDNA. Quantitation of signals indicated a 20- to 50-fold increase in signal with RCA. A robust signal was generated for each of the capture probes (representing different mRNAs) while direct label detection gave little or no signal for one third of the capture probes and only a weak signal for many of the other capture probes.

Assays were also performed using varied amounts of biotin-tagged cDNA applied to the microarrays during hybridization (cDNA prepared from 1.0 μg, 0.3 μg or 0.1 μg of mRNA from fibroblasts was applied to each subarray). The results showed a dose-response for RCA detection of each of the capture probes. That is, the signal intensity was correlated with the amount of mRNA used. The hybridized targets were detected by RCA signal amplification as described above. The results showed at least 10-fold increased sensitivity of detection of hybridized targets using RCA signal amplification as compared to direct detection of Cy5-labeled cDNA.

### H. Example 8

Signal Amplification of mRNA from Jurkat Cell Line

RNA from steady state Jurkat cells was processed as in Example 6. The results showed a signal increase with immunoRCA and negligible non-specific background. Quantitation of the fluorescence spots showed 20- to 50-fold greater signal intensity with RCA as compared to Cy5-labeled cDNA and up to a 1,000-fold signal increase with RCA.

### I. Example 9

Signal Amplification with Biotin Capture Tags Incorporated into cDNA Strands and BrdU Detection Labels Incorporated into TS-DNA

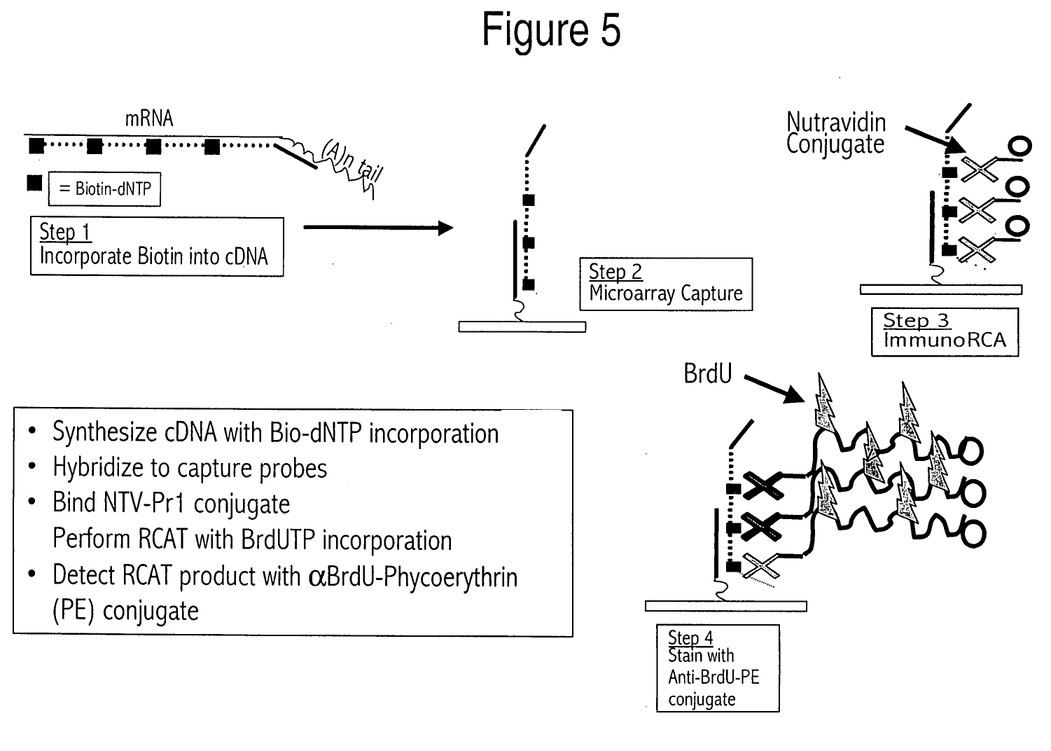

Microarrays were hybridized with 0.1 nM MSI-403 and MSI-405 target (Example 4). Amplification assays were performed generally as described in earlier examples using the scheme shown in FIG. 5. Biotin was incorporated into the targets (see Example 4). RCA was performed with BrdU incorporation into the tandem sequence DNA. RCA signal intensity was compared with signal intensity of direct detection of biotin tags on targets with streptavidin-phycoerythrin (SA-PE). The result was a 40- to 120-fold increase in signal from RCA compared with direct detection. The fold amplification is the ratio of mean fluorescence intensity with RCA over the mean fluorescence intensity with SA-PE.

### Illustrations

A. Illustration 1: FIG. 1

In this embodiment of the disclosed method, the RT primer is coupled to a 5′-terminal biotin moiety. Coupling may be covalent or non-covalent. For covalent coupling, the biotin moiety may be attached to the oligonucleotide via a linker, such as a carbon linker of 3 (C3), 6, 7, 12, 18 or more carbon residues. C3 linker is preferred. This embodiment is equally adaptable to the biotin moiety being linked at the 5′-end, the 3′-end or internally in the nucleotide sequence backbone of the RT primer. Other haptens, such as digoxigenin, may also be coupled to the RT primer. Numerous examples of non-covalent interactions between a ligand and its receptor have been described in the literature. This embodiment is generally compatible with most or all of those interactions. Typical examples of non-covalent interactions are DNA-protein interactions, protein-protein interactions, ligand-receptor interactions, enzyme-substrate interactions, and so on.

cDNA molecules produced in the reverse transcription step are hybridized to capture probes immobilized on an array. After stringent washes to remove non-specifically hyridized targets, the microarrays are incubated with anti-biotin antibody conjugate or Neutravidin conjugated with an RCA primer. RCA amplification is performed and the RCA product is detected by hybridizing detection probes (for example, short oligonucleotides coupled to a detectable tag, such as a fluorescence tag), and measuring the amount of fluorescence present at each spot on the microarray containing a capture probe.

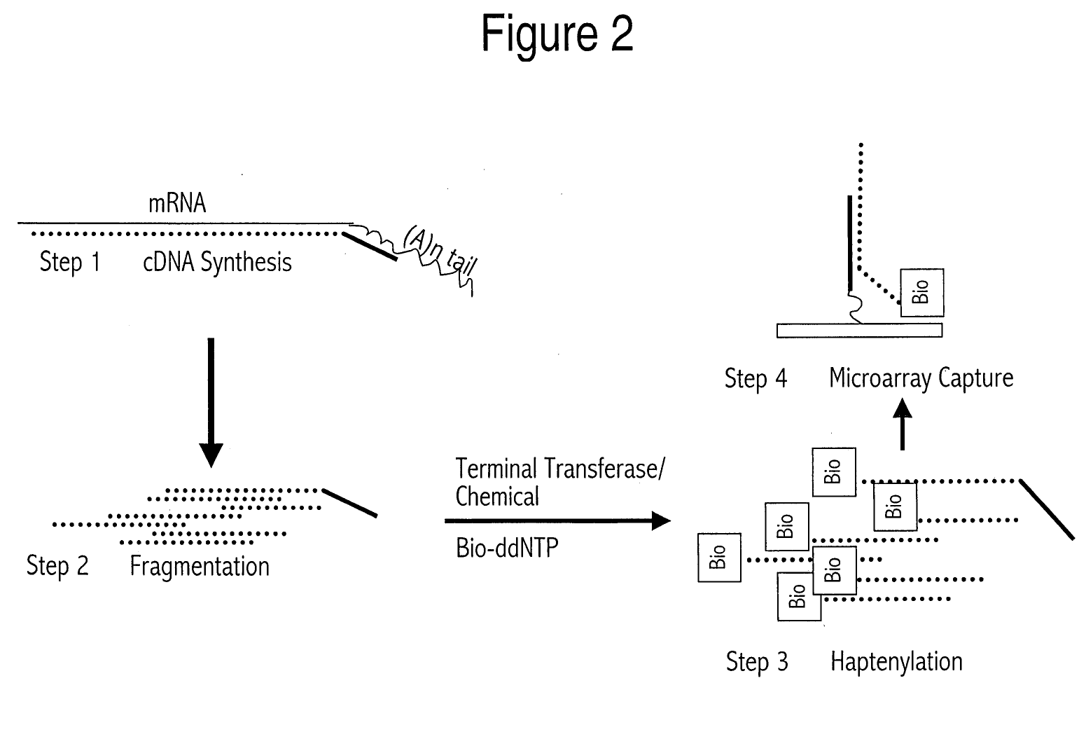

B. Illustration 2: FIG. 2

In this embodiment of the disclosed method, cDNA is synthesized by priming the reverse transcription reaction generally with an RT primer lacking a capture tag. The cDNA is then fragmented. Fragmentation may be achieved by a variety of means, and methods to do so have been described in the art. Treatment of single or double-stranded DNA molecules with sodium hydroxide solution has been shown to be an effective means of fragmentation. The average lengths of the products of the fragmentation can be controlled by varying either the concentration or the time of incubation with the sodium hydroxide solution. Alternative methods of fragmentation may also be employed. For instance, the enzyme Uracil-N-glycosylase may be employed in order to fragment cDNAs synthesized in the presence of dUTP. Another example of a fragmentation method is the use of deoxyribonucleases, such as DNAse I. The fragmented DNA molecules are then extended by a single nucleotide containing an attached capture tag (for example, a hapten molecule, such as biotin). This is achieved enzymatically by treating the DNA fragments with terminal transferase in presence of the biotinylated dideoxynucleotide.

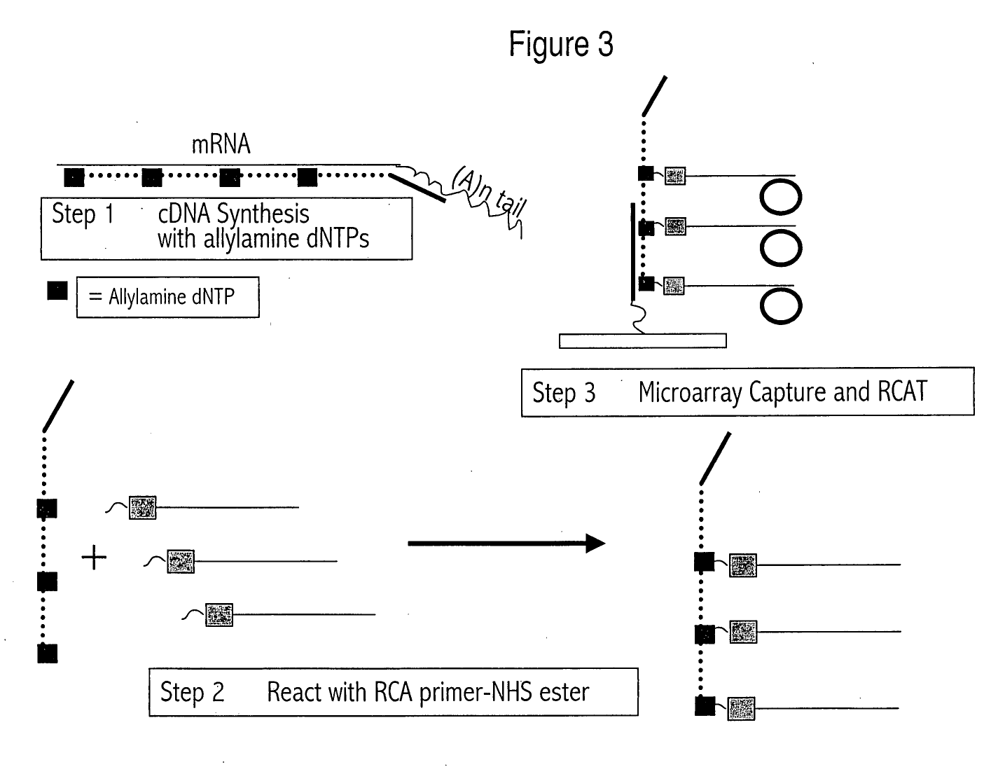

C. Illustration 3: FIG. 3

In this embodiment of the disclosed method, cDNA is synthesized in the presence of 5-(3-Aminoallyl)-2′-deoxyuridine 5′-triphosphate, sodium salt, (Sigma-Aldrich; Catalog A-0410) using established techniques. The cDNA synthesis reaction is the same as in Example 2, except that aadUTP is substituted for Cy5-dUTP as the capture tag incorporated into the cDNA strands, and oligo(dT)₁₈ is substituted for Biotin-oligo(dT)₁₈ as the RT primer.

After hydrolysis and clean-up of the cDNA as cDNA as described in the examples, a rolling circle replication primer containing an NHS ester as a capture tag is coupled to the cDNA fragments. The coupling (that is, association) is via the biotin capture tags incorporated into the cDNA strands and the NHS ester capture tag in the rolling circle replication primer. The cDNA pellet is resuspended in 9 μl 0.1 M sodium bicarbonate buffer (pH 9.0). The RCA primer is dissolved in 72 μl of 50% DMSO at a concentration of 1 μM. The allylamine labeled cDNA is mixed with the RCA primer and allowed to incubate for 1 hour at RT in the dark. Following the incubation 4.5 μl of 4 M hydroxylamine is added and incubated for a further 15 minutes at RT in the dark. Add 70 μl water and purify on a QiaQuick column as described above.

The rolling circle replication primer-tagged cDNA is hybridized to capture probes and RCA is performed as described above.

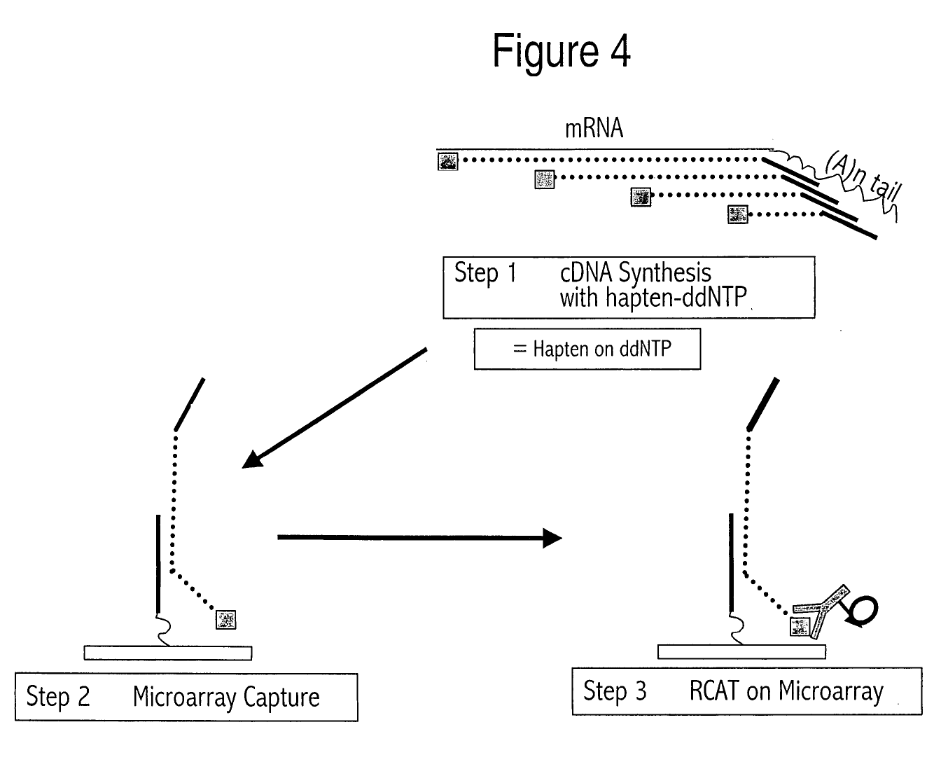

D. Illustration 4: FIG. 4

In this embodiment of the disclosed method, cDNA synthesis is performed in the presence of at least one dideoxynucleotide triphosphate tagged with a capture probe (for example, a detectable hapten, such as biotin). If only one species of dideoxynucleotide, such as ddUTP, is used in the reaction, the other nucleotides are supplemented as deoxynucleotide triphosphates. Typically, a mixture of Biotin-ddUTP and TTP is used in combination with the other nucleotide triphosphates. Preferred reverse transcriptase enzymes are RetroTherm or MMLV (Epicentre Technologies, Madison, Wis.), AMV (Amersham Pharmacia Biotech, Piscataway, N.J.) or SuperScript II (BRL, Bethesda, Md.). The biotin tagged cDNA is hybridized to capture probes on arrays and amplified by RCA as described above with the capture probe terminating the cDNA strand mediating association of the rolling circle replication primer.

E. Illustration 5: FIG. 5

In this embodiment of the disclosed method, cDNA synthesis is performed with incorporation of biotin-dUTP as described in Example 2 above for Cy5-cDNA synthesis. Biotin-tagged cDNAs are hybridized to capture probes on microarrays and incubated with Nutravidin-Primer1 conjugate (NTV conjugate). The NTV conjugate recognizes and binds to the biotin on the hybridized cDNA targets. In the next step, RCA is performed with Circle 1 in the presence of BrdUTP, so that BrdUrd is incorporated into the RCA product. After washing, the RCA product is detected with anti-BrdU antibody that is conjugated to a fluorophore, such as phycoerythrin (PE). Biotin is the capture tag in the cDNA strands, neutravidin is the capture tag in the rolling circle replication primer, and BrdU is the detection label incorporated into the tandem sequence DNA produced by rolling circle replication. Association of the rolling circle replication primers is via the capture tags in the cDNA strands and in the rolling circle replication primers.

It is understood that the disclosed invention is not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a host cell” includes a plurality of such host cells, reference to “the antibody” is a reference to one or more antibodies and equivalents thereof known to those skilled in the art, and so forth.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not. For example, the phrase “optionally substituted lower alkyl” means that the lower alkyl group may or may not be substituted and that the description includes both unsubstituted lower alkyl and lower alkyl where there is substitution.

Unless defined otherwise, all technical and scientific terms used herein have the same meanings as commonly understood by one of skill in the art to which the disclosed invention belongs. Although any methods and materials similar or equivalent to those described herein can be used in the practice or testing of the present invention, the preferred methods, devices, and materials are as described. Publications cited herein and the material for which they are cited are specifically incorporated by reference. Nothing herein is to be construed as an admission that the invention is not entitled to antedate such disclosure by virtue of prior invention.

Those skilled in the art will recognize, or be able to ascertain using no more than routine experimentation, many equivalents to the specific embodiments of the invention described herein. Such equivalents are intended to be encompassed by the following claims.

We claim:

1. A method of amplifying messenger RNA, the method comprising
(a) mixing one or more RT primers with a nucleic acid sample and reverse transcribing to produce cDNA strands each comprising one of the RT primers, wherein each RT primer comprises a reverse transcription primer portion,
(b) mixing the cDNA strands with a set of capture probes under conditions that promote hybridization of the cDNA strands to the capture probes,
(c) mixing one or more rolling circle replication primers with the cDNA strands under conditions that promote association of the cDNA strands with the rolling circle replication primers, wherein the rolling circle replication primers each comprise a capture tag, and wherein the association occurs via the capture tag,
(d) mixing one or more amplification target circles with the rolling circle replication primers under conditions that promote association of the rolling circle replication primers with the amplification target circles,
(e) incubating the amplification target circles under conditions that promote replication of the amplification target circles,
wherein replication of the amplification target circles results in the formation of tandem sequence DNA.

2. The method of claim 1 wherein the capture tag associates with the RT primer.

3. The method of claim 1 wherein the reverse transcription primer portion of each RT primer comprises poly T.

4. The method of claim 1 wherein the capture probes are immobilized on a substrate.

5. The method of claim 4 wherein the capture probes are in an array.

6. The method of claim 4 wherein the capture probes are immobilized via a capture tag coupled to the capture probes.

7. The method of claim 1 wherein each capture probe comprises a sequence matching all or a portion of the sequence of messenger RNA molecules of interest.

8. The method of claim 7 wherein the set of capture probes collectively comprise sequence matching all or a portion of the sequence of a plurality of different messenger RNA molecules of interest.

9. The method of claim 8 wherein the plurality of different messenger RNA molecules of interest comprise a set of messenger RNA molecules derived from, or present in, cells from a source of interest.

10. The method of claim 9 wherein the plurality of different messenger RNA molecules are associated with a condition or disease state of the cells or the source of interest.

11. The method of claim 8 wherein the plurality of different messenger RNA molecules of interest comprise a set of messenger RNA molecules representing a catalog of messenger RNA molecules from a source of interest.

12. The method of claim 8 wherein the plurality of different messenger RNA molecules of interest comprise a set of messenger RNA molecules from a plurality of sources of interest.

13. The method of claim 1 wherein the ends of the capture probes are extendable when a cDNA strand is hybridized to the capture probe.

14. The method of claim 13 wherein the ends of the capture probes are designed to be extendable only when a cDNA strand corresponding to a particular form of a messenger RNA of interest is hybridized to the capture probe.

15. The method of claim 1 wherein the ends of the capture probes are not extendable by polymerase.

16. The method of claim 1 further comprising, prior to step (c),
mixing one or more half probes with the cDNA strands, wherein each half probe is designed to hybridize to a cDNA strand adjacent to where a capture probe hybridizes,
ligating half probes and capture probes hybridized to cDNA strands.

17. The method of claim 16 further comprising, following ligation,
incubating the capture probes at a temperature above the melting temperature of the capture probe but below the melting temperature of the ligated capture probe/half probe.

18. The method of claim 1 further comprising, simultaneous with, or following, step (d),
mixing a secondary DNA strand displacement primer with the amplification target circles and incubating under conditions that promote hybridization between the tandem sequence DNA and the secondary DNA strand displacement primer and replication of the tandem sequence DNA,
wherein replication of the tandem sequence DNA results in the formation of secondary tandem sequence DNA.

19. The method of claim 18 further comprising, simultaneous with step (e),
mixing a tertiary DNA strand displacement primer with the amplification target circles.

20. The method of claim 1 further comprising detecting the tandem sequence DNA, wherein detection of tandem sequence DNA indicates that the corresponding messenger RNA molecule was present in the nucleic acid sample.

21. The method of claim 20 wherein the tandem sequence DNA is detected while in association with the capture probes.

22. The method of claim 21 wherein the identity of the capture probe associated with a tandem sequence DNA indicates the identity of the corresponding messenger RNA molecule.

23. The method of claim 21 wherein the tandem sequence DNA is detected at the site where the capture probe is located, and wherein the location of the capture probe indicates the identity of the corresponding messenger RNA molecule.

24. The method of claim 20 wherein detection is mediated by detection probes or by a detection label incorporated in the tandem sequence DNA.

25. The method of claim 24 wherein the detection label is a ligand.

26. The method of claim 25 wherein the ligand is biotin or BrdU.

27. The method of claim 26 wherein the ligand is BrdU, wherein the tandem sequence DNA is detected by associating an anti-BrdU antibody with the tandem sequence DNA and detecting the anti-BrdU antibody.

28. The method of claim 27 wherein the anti-BrdU antibody comprises a label, wherein the anti-BrdU antibody is detected by detecting the label.

29. The method of claim 28 wherein the label on the anti-BrdU antibody is a fluorophore.

30. The method of claim 28 wherein the fluorophore is phytoerythrin.

31. The method of claim 20 further comprising
mixing a set of detection probes with the tandem sequence DNA under conditions that promote hybridization between the tandem sequence DNA and the detection probes, and
detecting a plurality of different sequences present in the tandem sequence DNA.

32. The method of claim 1 wherein the tandem sequence DNA is collapsed using collapsing probes.

33. The method of claim 32 wherein at least one of the collapsing probes is a collapsing detection probe.

34. The method of claim 32 wherein the tandem sequence DNA is collapsed by mixing the collapsing probes with the tandem sequence DNA, and incubating under conditions that promote hybridization between the collapsing probes and the tandem sequence DNA.

35. The method of claim 34 further comprising, prior to or simultaneous with the mixing of the collapsing probes with the tandem sequence DNA, mixing detection probes with the tandem sequence DNA, and incubating under conditions that promote hybridization between the detection probes and the tandem sequence DNA.

36. The method of claim 32 wherein the collapsing probes comprise ligands, haptens, or both coupled to or incorporated into oligonucleotides.

37. The method of claim 1 wherein the RT primer comprises a capture tag.

38. The method of claim 37 wherein the capture tag on the RT primer is selected from the group consisting of biotin, digoxigenin, bromodeoxyuridine, or other hapten.

39. The method of claim 37 wherein the cDNA strands comprise capture tags.

40. The method of claim 1 wherein the cDNA strands comprise capture tags.

41. The method of claim 40 wherein the capture tags on the cDNA strands are selected from the group consisting of biotin, digoxigenin, bromodeoxyuridine, or other hapten.

42. The method of claim 1 wherein the association is covalent.

43. The method of claim 1 wherein the association is non-covalent.

44. The method of claim 43 wherein the association occurs between a protein and a nucleic acid.

45. The method of claim 44 wherein the association occurs between two proteins.

46. The method of claim 41 wherein the capture tags on the cDNA strands are biotin.

47. The method of claim 46 wherein the capture tags on the rolling circle replication primers comprise antibodies that bind biotin.

48. A method of amplifying messenger RNA, the method comprising
(a) mixing one or more RT primers with a nucleic acid sample and reverse transcribing to produce cDNA strands each comprising one of the RT primers,
(b) fragmenting the cDNA strands to form fragmented cDNA,
(c) adding a capture tag to the fragmented cDNA,
(d) mixing the fragmented cDNA with a set of capture probes under conditions that promote hybridization of the fragmented cDNA to the capture probes,
(e) mixing one or more rolling circle replication primers with the fragmented cDNA under conditions that promote association of the fragmented cDNA with the rolling circle replication primers, and wherein the association occurs via the capture tag,
(f) mixing one or more amplification target circles with the rolling circle replication primers under conditions that promote association of the rolling circle replication primers with the amplification target circles,
(g) incubating the amplification target circles under conditions that promote replication of the amplification target circles,
wherein replication of the amplification target circles results in the formation of tandem sequence DNA.

49. The method of claim 48 wherein the rolling circle replication primers each comprise a capture tag.

50. The method of claim 49 wherein association of the rolling circle replication primers with the cDNA occurs via association of the capture tag added to the fragmented cDNA and the capture tag in the rolling circle replication primers.

51. The method of claim 48 wherein the capture tag is added to the fragmented cDNA by terminal transferase.

52. The method of claim 51 wherein the capture tag is biotinylated-ddNTP.

53. A method of amplifying messenger RNA, the method comprising
(a) mixing one or more RT primers with a nucleic acid sample and reverse transcribing to produce cDNA strands each comprising one of the RT primers, wherein each RT primer comprises a reverse transcription primer portion and a capture tag,
(b) mixing the cDNA strands with a set of capture probes under conditions that promote hybridization of the cDNA strands to the capture probes,
(c) mixing one or more rolling circle replication primers with the cDNA strands under conditions that promote association of the cDNA strands to the rolling circle replication primers, and wherein the association occurs through the capture tag,
(d) mixing one or more amplification target circles with the rolling circle replication primers under conditions that promote association of the rolling circle replication primers with the amplification target circles,
(e) incubating the amplification target circles under conditions that promote replication of the amplification target circles,
wherein replication of the amplification target circles results in the formation of tandem sequence DNA.

54. The method of claim 53 wherein the rolling circle replication primers each comprise a capture tag.

55. The method of claim 54 wherein association of the rolling circle replication primers with the cDNA occurs via association of the capture tag added to the cDNA and the capture tag in the rolling circle replication primers.

56. A method of amplifying messenger RNA, the method comprising
(a) mixing one or more RT primers with a nucleic acid sample and reverse transcribing to produce cDNA strands each comprising one of the RT primers, wherein each RT primer comprises a reverse transcription primer portion, wherein the cDNA comprises a capture tag,
(b) mixing the cDNA strands with a set of capture probes under conditions that promote hybridization of the cDNA strands to the capture probes,
(c) mixing one or more rolling circle replication primers with the cDNA strands under conditions that promote association of the cDNA strands with the rolling circle replication primers, and wherein the association occurs through the capture tag,
(d) mixing one or more amplification target circles with the rolling circle replication primers under conditions that promote association of the rolling circle replication primers with the amplification target circles,
(e) incubating the amplification target circles under conditions that promote replication of the amplification target circles,
wherein replication of the amplification target circles results in the formation of tandem sequence DNA.

57. The method of claim 56 wherein the rolling circle replication primers each comprise a capture tag.

58. The method of claim 57 wherein association of the rolling circle replication primers with the cDNA occurs via association of the capture tag incorporated into the cDNA and the capture tag in the rolling circle replication primers.

59. The method of 56 wherein the capture tag is derived from allyl amine dUTP.

60. The method of 59 wherein the amplification target circle hybridizes with a rolling circle amplification primer comprising an NHS ester.

61. The method of claim 57 wherein the capture tag is derived from incorporation of biotinylated-ddNTP into the cDNA.

62. A method of amplifying messenger RNA, the method comprising
(a) mixing one or more RT primers with a nucleic acid sample and reverse transcribing to produce cDNA strands each comprising one of the RT primers, wherein each RT primer comprises a reverse transcription primer portion and a rolling circle replication primer portion,
wherein the reverse transcription primer portion and the rolling circle replication primer portion each comprise a 5′ end, wherein the reverse transcription primer portion and the rolling circle replication primer portion are not linked via their 5′ ends,
(b) mixing the cDNA strands with a set of capture probes under conditions that promote hybridization of the cDNA strands to the capture probes,
(c) mixing one or more amplification target circles with the rolling circle replication primer portions under conditions that promote association of the rolling circle replication primer portions with the amplification target circles,
(d) incubating the amplification target circles under conditions that promote replication of the amplification target circles,
wherein replication of the amplification target circles results in the formation of tandem sequence DNA.

63. A kit for amplifying messenger RNA, the kit comprising,
(a) one or more amplification target circles,
wherein the amplification target circles each comprise a single-stranded, circular DNA molecule comprising a primer complement portion, and
(b) one or more RT primers,
wherein the RT primers each comprise a reverse transcription primer portion and a rolling circle replication primer portion,
wherein the reverse transcription primer portion and the rolling circle replication primer portion each comprise a 5′ end, wherein the reverse transcription primer portion and the rolling circle replication primer portion are not linked via their 5′ ends, wherein both the reverse transcription primer portion and the rolling circle replication primer portion can prime nucleic acid replication,
wherein the rolling circle replication primer portion is complementary to a portion of one or more amplification target circles, and
(c) one or more capture probes,
wherein each capture probe comprises a sequence matching all or a portion of the sequence of messenger RNA molecules of interest.

64. The kit of claim 63 wherein the reverse transcription primer portion of each RT primer comprises poly T.

65. The kit of claim 63 further comprising a secondary DNA strand displacement primer comprising a single-stranded, linear nucleic acid molecule comprising a matching portion that matches a portion of one or more of the amplification target circles.

66. The kit of claim 65 further comprising a tertiary DNA strand displacement primer comprising a single-stranded, linear nucleic acid molecule comprising a complementary portion that is complementary to a portion of one or more of the amplification target circles.

67. A mixture comprising
(a) cDNA strands produced by incubating one or more RT primers with a nucleic acid sample and reverse transcribing, wherein each cDNA strand comprises one of the RT primers, wherein each RT primer comprises a reverse transcription primer portion,
(b) a set of capture probes hybridized to the cDNA strands,
(c) one or more rolling circle replication primers associated with the cDNA strands, wherein the rolling circle replication primers each comprise a capture tag, and wherein the association occurs via the capture tag,
(d) one or more amplification target circles associated with the rolling circle replication primers.

68. A method of using messenger RNA, the method comprising
replicating one or more amplification target circles to produce one or more tandem sequence DNAs,
wherein each tandem sequence DNA is coupled to a rolling circle replication primer, wherein the rolling circle replication primer is associated with a cDNA strand, wherein the rolling circle replication primer comprises a capture tag, wherein the association occurs via the capture tag, wherein the cDNA strand is hybridized to a capture probe, wherein the cDNA strand comprises an RT primer, wherein the cDNA strand is produced by reverse transcribing a nucleic acid sample with the RT primer.

69. A method of using messenger RNA, the method comprising
replicating one or more amplification target circles to produce one or more tandem sequence DNAs,
wherein each tandem sequence DNA is coupled to a rolling circle replication primer, wherein the rolling circle replication primer is associated with a fragmented cDNA strand, wherein the fragmented cDNA strand is hybridized to a capture probe, wherein the fragmented cDNA comprises a capture tag, wherein the association occurs via the capture tag, wherein the fragmented cDNA strand is a fragment of a cDNA strand, wherein the cDNA strand comprises an RT primer, wherein the cDNA strand is produced by reverse transcribing a nucleic acid sample with the RT primer.

70. A method of using messenger RNA, the method comprising
replicating one or more amplification target circles to produce one or more tandem sequence DNAs,
wherein each tandem sequence DNA is coupled to a rolling circle replication primer, wherein the rolling circle replication primer is associated with a cDNA strand, wherein the cDNA strand is hybridized to a capture probe, wherein the cDNA strand comprises an RT primer, wherein the RT primer comprises a capture tag, wherein the association occurs via the capture tag, wherein the cDNA strand is produced by reverse transcribing a nucleic acid sample with the RT primer.

71. A method of using messenger RNA, the method comprising
replicating one or more amplification target circles to produce one or more tandem sequence DNAs,
wherein each tandem sequence DNA is coupled to a rolling circle replication primer, wherein the rolling circle replication primer is associated with a cDNA strand, wherein the cDNA strand comprises a capture tag, wherein the association occurs via the capture tag, wherein the cDNA strand is hybridized to a capture probe, wherein the cDNA strand comprises an RT primer, wherein the cDNA strand is produced by reverse transcribing a nucleic acid sample with the RT primer.

72. A method of using messenger RNA, the method comprising
replicating one or more amplification target circles to produce one or more tandem sequence DNAs,
wherein each tandem sequence DNA is coupled to a rolling circle replication primer portion of an RT primer that comprises the rolling circle replication primer portion and a reverse transcription primer portion, wherein the cDNA strand is hybridized to a capture probe, wherein the cDNA strand comprises the RT primer, wherein the cDNA strand is produced by reverse transcribing a nucleic acid sample with the RT primer,
wherein the reverse transcription primer portion and the rolling circle replication primer portion each comprise a 5′ end, wherein the reverse transcription primer portion and the rolling circle replication primer portion are not linked via their 5′ ends.

73. A method of amplifying messenger RNA, the method comprising
(a) mixing one or more RT primers with a nucleic acid sample and reverse transcribing to produce cDNA strands each comprising one of the RT primers, wherein each RT primer comprises a reverse transcription primer portion, wherein the cDNA strands comprise capture tags, wherein the capture tags on the cDNA strands are biotin,
(b) mixing the cDNA strands with a set of capture probes under conditions that promote hybridization of the cDNA strands to the capture probes,
(c) mixing one or more rolling circle replication primers with the cDNA strands under conditions that promote association of the cDNA strands with the rolling circle replication primers, wherein the rolling circle replication primers each comprise a capture tag, wherein the capture tags on the rolling circle replication primers comprise antibodies that bind biotin, wherein the association occurs via the capture tags on the cDNA strands and the capture tags on the rolling circle replication primers,
(d) mixing one or more amplification target circles with the rolling circle replication primers under conditions that promote association of the rolling circle replication primers with the amplification target circles,
(e) incubating the amplification target circles under conditions that promote replication of the amplification target circles,
wherein replication of the amplification target circles results in the formation of tandem sequence DNA,
(f) detecting the tandem sequence DNA, wherein detection of tandem sequence DNA indicates that the corresponding messenger RNA molecule was present in the nucleic acid sample,
wherein detection is mediated by a detection label incorporated in the tandem sequence DNA, wherein the detection label is BrdU,
wherein the tandem sequence DNA is detected by associating an anti-BrdU antibody with the tandem sequence DNA and detecting the anti-BrdU antibody,
wherein the anti-BrdU antibody comprises a label, wherein the label is phytoerythrin, wherein the anti-BrdU antibody is detected by detecting the label.


SEQUENCE LISTING (27 sequences)

SEQ ID NO: 1 - DNA, 34 bp, Artificial Sequence
  Description of Artificial Sequence; Note = synthetic construct
aaaaaaaaaaaaaaaccagaagagatgtctgtgg

SEQ ID NO: 2 - DNA, 35 bp, Artificial Sequence
  Description of Artificial Sequence; Note = synthetic construct
aaaaaaaaaaaaaaaggtgatgacttagcgtcaag

SEQ ID NO: 3 - DNA, 37 bp, Artificial Sequence
  Description of Artificial Sequence; Note = synthetic construct
aaaaaaaaaaaaaaagtttaaaaagtttcacgtcttg

SEQ ID NO: 4 - DNA, 34 bp, Artificial Sequence
  Description of Artificial Sequence; Note = synthetic construct
aaaaaaaaaaaaaaactgcaggacatgacaactc

SEQ ID NO: 5 - DNA, 38 bp, Artificial Sequence
  Description of Artificial Sequence; Note = synthetic construct
aaaaaaaaaaaaaaagtaattaggaacctgtttcttac

SEQ ID NO: 6 - DNA, 33 bp, Artificial Sequence
  Description of Artificial Sequence; Note = synthetic construct
aaaaaaaaaaaaaaacttctgaacgtcccctgc

SEQ ID NO: 7 - DNA, 38 bp, Artificial Sequence
  Description of Artificial Sequence; Note = synthetic construct
aaaaaaaaaaaaaaagaagatgaatcattgattgaata

SEQ ID NO: 8 - DNA, 36 bp, Artificial Sequence
  Description of Artificial Sequence; Note = synthetic construct
aaaaaaaaaaaaaaacgttaacacaaaatccatggg

SEQ ID NO: 9 - DNA, 35 bp, Artificial Sequence
  Description of Artificial Sequence; Note = synthetic construct
aaaaaaaaaaaaaaaacgacaaaaggagcttttgc

SEQ ID NO: 10 - DNA, 34 bp, Artificial Sequence
  Description of Artificial Sequence; Note = synthetic construct
aaaaaaaaaaaaaaaggtctcaaagaggaagagc

SEQ ID NO: 11 - DNA, 36 bp, Artificial Sequence
  Description of Artificial Sequence; Note = synthetic construct
aaaaaaaaaaaaaaagctctaggaagacatttttcc

SEQ ID NO: 12 - DNA, 36 bp, Artificial Sequence
  Description of Artificial Sequence; Note = synthetic construct
aaaaaaaaaaaaaaaccagagagaatatccagagat

SEQ ID NO: 13 - DNA, 35 bp, Artificial Sequence
  Description of Artificial Sequence; Note = synthetic construct
aaaaaaaaaaaaaaaccatgtgatgctcaatggat

SEQ ID NO: 14 - DNA, 35 bp, Artificial Sequence
  Description of Artificial Sequence; Note = synthetic construct
aaaaaaaaaaaaaaagatttccaacatcctgcagg

SEQ ID NO: 15 - DNA, 36 bp, Artificial Sequence
  Description of Artificial Sequence; Note = synthetic construct
aaaaaaaaaaaaaaacaagtttaaggagaagctgac

SEQ ID NO: 16 - DNA, 38 bp, Artificial Sequence
  Description of Artificial Sequence; Note = synthetic construct
aaaaaaaaaaaaaaagattctaagagctttaaactttg

SEQ ID NO: 17 - DNA, 36 bp, Artificial Sequence
  Description of Artificial Sequence; Note = synthetic construct
aaaaaaaaaaaaaaacagtttaatggacactaagtc

SEQ ID NO: 18 - DNA, 37 bp, Artificial Sequence
  Description of Artificial Sequence; Note = synthetic construct
aaaaaaaaaaaaaaactacttatactggttcataatc

SEQ ID NO: 19 - DNA, 40 bp, Artificial Sequence
  Description of Artificial Sequence; Note = synthetic construct
tttggaaccagcgcagtgttgacaggtacaagaaccagtt

SEQ ID NO: 20 - DNA, 40 bp, Artificial Sequence
  Description of Artificial Sequence; Note = synthetic construct
tttggaaccagcgcagtgttgacaggtacaagaaccagta

SEQ ID NO: 21 - DNA, 38 bp, Artificial Sequence
  Description of Artificial Sequence; Note = synthetic construct
gaactatattgtctttctctgcaaacttggagatgtcc

SEQ ID NO: 22 - DNA, 38 bp, Artificial Sequence
  Description of Artificial Sequence; Note = synthetic construct
gaactatattgtctttctctgcaaacttggagatgtcg

SEQ ID NO: 23 - DNA, 40 bp, Artificial Sequence
  Description of Artificial Sequence; Note = synthetic construct
ggacatctccaagtttgcagagaaagacaatatagttctt

SEQ ID NO: 24 - DNA, 40 bp, Artificial Sequence
  Description of Artificial Sequence; Note = synthetic construct
aactggttcttgtacctgtcaacactgcgctggttccaaa

SEQ ID NO: 25 - DNA, 80 bp, Artificial Sequence
  Description of Artificial Sequence; Note = synthetic construct
ctcagctgtgtaacaacatgaagattgtaggtcagaactcacctgttagaaactgtgaagatcgcttattatgtcctatc

SEQ ID NO: 26 - DNA, 78 bp, Artificial Sequence
  Description of Artificial Sequence; Note = synthetic construct
aaaaaaaaaaaaaaaaaaaaaaaaaaaaaaaaaaaaaaaaaaaaaaaaaaacacagctgaggataggacataataagc

SEQ ID NO: 27 - DNA, 18 bp, Artificial Sequence
  Description of Artificial Sequence; Note = synthetic construct
tgtcctatcctcagctgg